(19) United States
(12) Patent Application Publication
(10) Pub. No.: US 2025/0266879 A1
(43) Pub. Date: Aug. 21, 2025

(54) CHANNEL STATE INFORMATION REFERENCE SIGNAL ANTENNA PORTS ADAPTATION IN WIRELESS COMMUNICATIONS SYSTEMS

(71) Applicant: APPLE INC., Cupertino, CA (US)

(72) Inventors: Hong He, San Jose, CA (US); Jie Cui, San Jose, CA (US); Dawei Zhang, Saratoga, CA (US); Wei Zeng, San Diego, CA (US); Haitong Sun, Cupertino, CA (US); Yushu Zhang, Beijing (CN); Chunhai Yao, Beijing (CN); Sigen Ye, San Diego, CA (US); Chunxuan Ye, San Diego, CA (US); Huaning Niu, San Jose, CA (US)

(21) Appl. No.: 18/859,236

(22) PCT Filed: Apr. 28, 2023
(86) PCT No.: PCT/US2023/066332

§ 371 Date: Oct. 23, 2024

Related U.S. Application Data
(60) Provisional application No. 63/363,821, filed on Apr. 29, 2022.

(51) Int. Cl. H04B 7/06; H04L 1/1812; H04L 5/00; H04W 24/10

(52) U.S. Cl. CPC H04B 7/0626; H04L 1/1812; H04L 5/0048; H04W 24/10

(57) ABSTRACT

Systems and methods for network power savings attendant to the performance of channel state information (CSI) reporting are disclosed herein. A base station may configure multiple periodic CSI (P-CSI) report configurations to a UE, and then dynamically indicate to the UE which of the P-CSI report configurations is to be used. A base station may configure a CSI report (e.g., a P-CSI report configuration and/or an aperiodic CSI (A-CSI) configuration) having multiple sub-configurations to a UE, and then dynamically indicate to the UE which of the sub-configurations is to be used. A base station may configure an M-bit CSI-RS bitmap indicating a set of active CSI-reference signal (CSI-RS) ports at the base station, such that the UE may perform CSI measurement using those CSI-RS ports in an expected manner.

### TECHNICAL FIELD

This application relates generally to wireless communication systems, including wireless communications systems using CSI reporting processes.

### BACKGROUND

Wireless mobile communication technology uses various standards and protocols to transmit data between a base station and a wireless communication device. Wireless communication system standards and protocols can include, for example, 3rd Generation Partnership Project (3GPP) long term evolution (LTE) (e.g., 4G), 3GPP new radio (NR) (e.g., 5G), and IEEE 802.11 standard for wireless local area networks (WLAN) (commonly known to industry groups as Wi-Fi®).

As contemplated by the 3GPP, different wireless communication systems standards and protocols can use various radio access networks (RANs) for communicating between a base station of the RAN (which may also sometimes be referred to generally as a RAN node, a network node, or simply a node) and a wireless communication device known as a user equipment (UE). 3GPP RANs can include, for example, global system for mobile communications (GSM), enhanced data rates for GSM evolution (EDGE) RAN (GERAN), Universal Terrestrial Radio Access Network (UTRAN), Evolved Universal Terrestrial Radio Access Network (E-UTRAN), and/or Next-Generation Radio Access Network (NG-RAN).

Each RAN may use one or more radio access technologies (RATs) to perform communication between the base station and the UE. For example, the GERAN implements GSM and/or EDGE RAT, the UTRAN implements universal mobile telecommunication system (UMTS) RAT or other 3GPP RAT, the E-UTRAN implements LTE RAT (sometimes simply referred to as LTE), and NG-RAN implements NR RAT (sometimes referred to herein as 5G RAT, 5G NR RAT, or simply NR). In certain deployments, the E-UTRAN may also implement NR RAT. In certain deployments, NG-RAN may also implement LTE RAT.

A base station used by a RAN may correspond to that RAN. One example of an E-UTRAN base station is an Evolved Universal Terrestrial Radio Access Network (E-UTRAN) Node B (also commonly denoted as evolved Node B, enhanced Node B, eNodeB, or eNB). One example of an NG-RAN base station is a next generation Node B (also sometimes referred to as a g Node B or gNB).

A RAN provides its communication services with external entities through its connection to a core network (CN). For example, E-UTRAN may utilize an Evolved Packet Core (EPC), while NG-RAN may utilize a 5G Core Network (5GC).

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a table defining various groups of P-CSI report configurations that may be provided to a UE, according to an embodiment.

FIG. 2 illustrates various antenna array configurations as may correspond to various P-CSI report configurations that have been configured to a UE, according to an embodiment.

FIG. 3 illustrates a MAC CE for dynamically indicating a P-CSI report configuration to a UE, according to an embodiment.

FIG. 4 illustrates a DCI format for activating a P-CSI report configuration, according to an embodiment.

FIG. 5 illustrates a group-common DCI format for activating one or more P-CSI reporting configurations, according to an embodiment.

FIG. 6 illustrates a UE-specific DCI format that may be used for P-CSI report activation, according to an embodiment.

FIG. 7 illustrates a DCI format for triggering A-CSI, according to an embodiment.

FIG. 8 illustrates a method of a UE, according to an embodiment.

FIG. 9 illustrates a method of a base station, according to an embodiment.

FIG. 10 illustrates a P-CSI report configuration that lists various sub-configurations for providing CSI feedback, according to an embodiment.

FIG. 11 illustrates a MAC CE for dynamically indicating a sub-configuration of a P-CSI report configuration to a UE, according to an embodiment.

FIG. 12 illustrates a DCI format for activating a sub-configuration of a P-CSI report configuration, according to an embodiment.

FIG. 13 illustrates a group-common DCI format for activating one or more sub-configurations of a P-CSI report configuration, according to an embodiment.

FIG. 14 illustrates a UE-specific DCI format that may be used for activation of a sub-configuration of a P-CSI report configuration, according to an embodiment.

FIG. 15 illustrates a DCI format for triggering A-CSI, according to an embodiment.

FIG. 16 illustrates a method of a UE, according to an embodiment.

FIG. 17 illustrates a method of a base station, according to an embodiment

FIG. 18 illustrates a group-common DCI format for communicating one or more M-bit CSI-RS ports bitmap fields, according to an embodiment.

FIG. 19 illustrates an antenna panel NULA arrangement for a CSI feedback process, according to an embodiment.

FIG. 20 illustrates an antenna panel NULA arrangement for a CSI feedback process, according to an embodiment.

FIG. 21 illustrates a method of a UE, according to an embodiment.

FIG. 22 illustrates a method of a base station, according to an embodiment.

FIG. 23 illustrates an example architecture of a wireless communication system, according to embodiments disclosed herein.

FIG. 24 illustrates a system for performing signaling between a wireless device and a network device, according to embodiments disclosed herein.

### DETAILED DESCRIPTION

Various embodiments are described with regard to a UE. However, reference to a UE is merely provided for illustrative purposes. The example embodiments may be utilized with any electronic component that may establish a connection to a network and is configured with the hardware, software, and/or firmware to exchange information and data with the network. Therefore, the UE as described herein is used to represent any appropriate electronic component.

In various wireless communications networks, it may be beneficial to implement methods for power saving at the network side (e.g., at a base station of the RAN). Accordingly, techniques may be developed for and implemented on base stations and/or UEs that allow for and/or facilitate power savings at the network. Such techniques may relate to achieving such reduced power conditions dynamically and/or semi-statically. Such techniques may use finer granularity adaptation of transmissions and/or receptions as related to one or more of the time domain, the frequency domain, the spatial domain, and/or the power domain. Support/feedback from the UE to the base station (including, for example, the use of UE assistance information) is contemplated.

As related to the spatial domain, it is contemplated that improvements to channel state information (CSI) reporting may be applied in a wireless communications system in order to achieve desirable power savings at the network.

Various wireless communications networks may support different CSI reporting types. These reporting types may include, for example, a periodic CSI (P-CSI) reporting type, a semi-persistent (SP) CSI (SP-CSI) reporting type, and an aperiodic CSI (A-CSI) reporting type.

It may be that in some such networks, for periodic CSI reporting, a maximum number of CSI reference signal (CSI-RS) resource sets that may be configured is limited to S=1, and this (one) CSI-RS resource set may use one or more CSI-RS resources, where each such CSI-RS resource is configured with a same number of antenna ports M (where M is up to 32).

However, as discussed herein, that other approaches may be used that provide a network with additional flexibility. For example, approaches herein may allow the network to more quickly adapt its use of a current CSI reporting style in order to match an appropriate complexity for current network conditions. Thus, as network conditions may allow, the network is enabled to save power by dynamically and relatively quickly selecting to less complex CSI reporting arrangements.

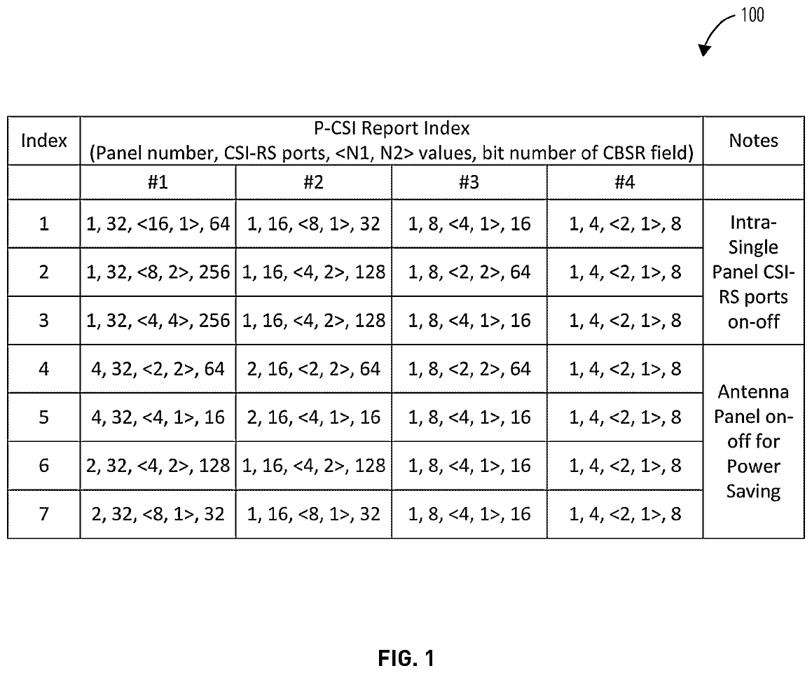

FIG. 1 illustrates a table 100 defining various groups of P-CSI report configurations that may be provided to a UE, according to an embodiment. The table 100 may correspond to a two-step P-CSI report selection process.

As can be seen in reference to FIG. 1, the groups of P-CSI report configurations are indexed from 1 to 7 in the table 100, and each includes four P-CSI report configurations. Each P-CSI report configuration defines a manner in which a CSI-RS to be used to generate a P-CSI report may be transmitted from the base station to the UE. It is noted that the number of groups of P-CSI report configurations known at the base station, the number of P-CSI report configurations that make up each group, and the contents of individual P-CSI report configurations that make up each group as illustrated in the table 100 are given by way of example (and not by way of limitation).

A group of P-CSI report configurations (e.g., one of the groups represented by indexes 1-7) may be formulated by the base station for use with a component carrier (CC) used between the UE and the base station. Then, the base station may transmit the P-CSI report configurations of the formulated group to the UE. In this way, the UE is informed of the group of P-CSI report configurations that the base station may be expected to use for P-CSI report adaptation for the CC going forward. For example, if the base station formulates the group of P-CSI report configurations represented by index 1 in the table 100, the base station would then transmit the four P-CSI report configurations corresponding to index 1 to the UE. In some cases, the base station may so provide the group of P-CSI report configurations to the UE via radio resource control (RRC) signaling.

It is noted that while the table 100 is provided here for clarity of discussion, a base station may not actually store/use a table like the table 100 to formulate a group of P-CSI report configurations (e.g., the base station may formulate the P-CSI report configurations to send to the UE in a manner other than by reference to, for example, any indexed table of pre-established groups of P-CSI report configurations).

Each P-CSI report configuration in a group is configured with various parameters. For example, it may be that each P-CSI report configuration in the group that is provided to the UE has a separate CSI-RS resource set configuration. It may be that each of these separate CSI-RS resource set configurations is for a different number of CSI-RS ports to be used for a corresponding CSI-RS resource of the set. For example, in the group represented by index 1 in the table 100, it may be seen that the first P-CSI report configuration (represented by “#1”) is for 32 CSI-RS ports, the second P-CSI report configuration (represented by “#2”) is for 16 CSI-RS ports, the third P-CSI report configuration (represented by “#3”) is for 8 CSI-RS ports, and the fourth P-CSI report configuration (represented by “#4”) is for 4 CSI-RS ports.

Further, the one or more P-CSI report configurations in a group may include a plurality of antenna panel number configurations. For example, in the group represented by index 4 in the table, the first P-CSI report configuration is for the use of four antenna panels, the second P-CSI report configuration is for the use of 2 antenna panels, and the third and fourth P-CSI report configurations are for the use of 1 antenna panel. As is noted in the table 100, the groups of P-CSI configurations represented by indexes 1-3 do not have P-CSI report configuration(s) for more than one antenna panel, while the groups of P-CSI configurations represented by indexes 4-7 do.

The one or more P-CSI report configurations in a group may include a plurality of antenna array configurations. Such configurations may define the arrangement of the antennas of the one or more antenna panels called for in the P-CSI report configurations. This may be done by indicating the number of antenna elements in horizontal direction ‘N1’ and the number of antenna elements in vertical direction ‘N2’ in an <N1, N2> pair. It is noted that each such antenna element may be cross-polarized such that it corresponds to 2 CSI-RS ports of a CSI-RS resource for the given P-CSI report configuration.

The one or more P-CSI report configurations in a group may include a plurality of codebook subset restriction (CSBR) bitmap configurations. For example, in the group represented by index 1 in the table, the first P-CSI report configuration is for the use of a bitmap configuration having 64 bits, the second P-CSI report configuration is for the use of bitmap configuration having 32 bits, the third and fourth P-CSI report configurations are for the use of a bitmap configuration having 16 bits, and the fourth P-CSI report configuration is for the use of a bitmap configuration having 8 bits.

The one or more P-CSI report configurations in a group may include a plurality of reporting periodicity configurations (not illustrated in the table 100).

The one or more P-CSI report configurations in a group may include a plurality of frequency granularity configurations (not illustrated in the table 100). For example, the P-CSI report configurations in a group may represent a plurality of frequency granularities for channel quality index (CQI) and/or precoder matrix indicators (PMIs) (e.g., wideband versus subband).

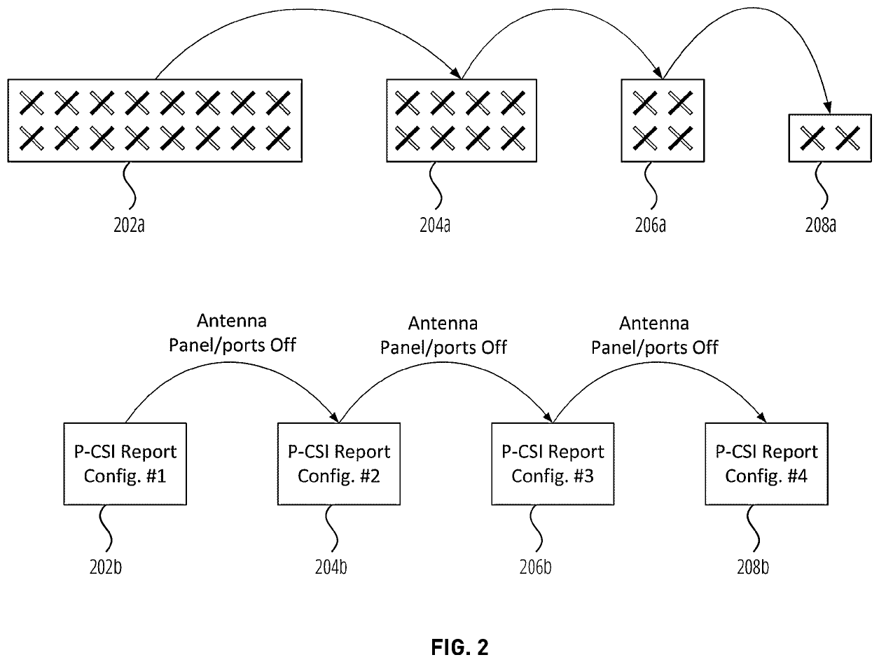

In some designs for groups of P-CSI report configurations, it may be that the supported antenna port layouts for on-off operation may be limited depending on the antenna array configuration of the ‘maximum’ CSI-RS ports number found in the group. FIG. 2 illustrates various antenna array configurations 202a through 208a as may correspond to a group of P-CSI report configurations 202b through 208b that have been configured to a UE, according to an embodiment. In the embodiment illustrated in FIG. 2, the first antenna array configuration 202a corresponds to a first P-CSI report configuration 202b, a second antenna array configuration 204a corresponds to a second P-CSI report configuration 204b, a third antenna array configuration 206a corresponds to a third P-CSI report configuration 206b, and a fourth antenna array configuration 208a corresponds to a fourth P-CSI report configuration 208b.

In the embodiment of FIG. 2, the P-CSI report configurations 202b through 208b may be the group of P-CSI report configurations represented by index 2 of the table 100 of FIG. 1. Accordingly, as can be seen, the antenna array configurations 202a through 208a match the antenna array configurations indicated in those CSI reports.

With reference to index 2 of the table 100 of FIG. 1, it can be determined that the first P-CSI report configuration (e.g., the first P-CSI report configuration 202b) having the maximum CSI-RS ports number (32) has an antenna array configuration of <8, 2>. Accordingly, it may be expected that the second P-CSI report configuration 204b, the third P-CSI report configuration 206b, and the fourth P-CSI report configuration 208b use antenna array configurations that are sized such that they fit within the dimension <8, 2>. For example, the second P-CSI report configuration 204b uses an antenna array configuration of <4, 2> (as illustrated by the second antenna array configuration 204a), the third P-CSI report configuration 206b uses an antenna array configuration of <2, 2> (as illustrated by the third antenna array configuration 206a), and the fourth P-CSI report configuration 208b uses an antenna array configuration of <2, 1>.

Accordingly, it can be seen that as the UE proceeds along through one or more of the first P-CSI report configuration 202b through the fourth P-CSI report configuration 208b, fewer overall antenna elements are needed for the transmission of a CSI-RS of a particular P-CSI report configuration (and thus less power may be used at the base station).

A group of P-CSI report configurations sent to a UE may include a same configuration for some parameters, such as, for example, a “reportQuantity” parameter.

Once a group of P-CSI report configurations is provided/configured to the UE by the base station, the base station may the provide signaling to the UE to dynamically indicate which P-CSI report configuration of the group is being used for the transmission of a CSI-RSs by the base station. This gives the base station flexibility, in that it may dynamically and relatively quickly, for example, move the CSI reporting process between the base station and the UE from a higher complexity P-CSI reporting configuration to a lower complexity P-CSI reporting configuration (and thus achieve power savings at the network), as network conditions may permit.

A variety of methods for providing the UE with a signaling indication of a P-CSI report configuration to use are considered. In a first case, an indication of the P-CSI report configuration may be sent in a medium access control control element (MAC CE). Such a MAC CE may be identified by a medium access control (MAC) subheader with a dedicated logical channel identifier (LCID) for MAC CE that include such indications. The MAC CE may have a fixed size.

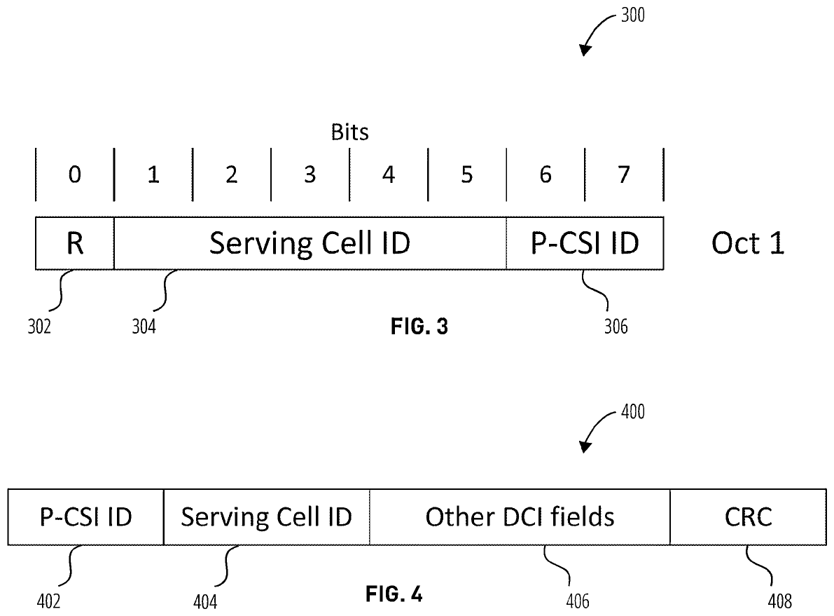

FIG. 3 illustrates a MAC CE 300 for dynamically indicating a P-CSI report configuration to a UE, according to an embodiment. The MAC CE 300 may include a number of octets N that together contain a pair of information elements (IEs). In the embodiment of FIG. 3, N=1 (other cases are contemplated). In the MAC CE 300, there is one reserved bit 302 (bit 0), five bits (bits 1 through 5) are used for a serving cell identifier (ID) IE 304 that indicates the identity of the serving cell for which this MAC CE is providing P-CSI report configuration indication, and two bits (bits 6 and 7) are used for a P-CSI ID IE 306 to indicate the particular P-CSI report configuration that should be used on the CC of the identified serving cell.

The use of two bits for the P-CSI ID IE 306 may correspond to the case where the group of P-CSI report configurations previously configured to the UE is four (where the four P-CSI configurations can be uniquely represented using two bits). More generally, it may be understood that a number of bits needed for a P-CSI ID IE may be log₂(M) when M represents the total number of P-CSI configurations that have been provided to the UE by the base station. Then, to indicate a particular P-CSI report configuration, the P-CSI ID IE 306 may indicate a value k that corresponds to the k^{th} sub-configuration. Accordingly, in cases where many P-CSI configurations are configured to a UE by the base station, the MAC CE may use more than N=1 octets in order to accommodate the bits of an appropriately-sized P-CSI ID IE.

In some cases, the P-CSI report configuration given by a MAC CE may be activated at a time instance n+T_{proc}. In some cases, n may be either a slot of a physical downlink shared channel (PDSCH) containing the MAC CE or a slot of a physical uplink control channel (PUCCH) containing hybrid automatic repeat request acknowledgement (HARQ-ACK) signaling for the PDSCH.

Further, T_{proc} may be a processing time for applying the first P-CSI report configuration at the UE. In some cases, the value of T_{proc} may be pre-established by a specification that defines the behavior of the wireless communication system (e.g., the specification may define that T_{proc}=3 milliseconds (ms)). In some cases, it may be that such a specification provides a set of candidate values for T_{proc}, and that the UE then indicates an appropriate/desired T_{proc} (e.g., corresponding to a UE capability) to the base station via UE capability reporting/messaging.

In a second case of methods for providing the UE with a signaling indication of a P-CSI report configuration, a downlink control information (DCI) format for activating/deactivating particular P-CSI report configuration(s) from the group of P-CSI report configurations configured to the UE may be used.

FIG. 4 illustrates a DCI format 400 for activating a P-CSI report configuration, according to an embodiment. The DCI format 400 may represent, for example, a non-fallback scheduling DCI of a (modified) format of X_1 and or X_2, as may be understood in some specifications for some wireless communications systems.

A P-CSI ID field 402 of the DCI format 400 may indicate a particular P-CSI report configuration that should be activated at the receiving UE for P-CSI reporting.

In at least some cases, the serving cell ID field 404 may include the serving cell ID field 404 as illustrated. A serving cell ID field 404 of the DCI format 400 may indicate the serving cell corresponding to the CC for which the particular P-CSI report configuration should be activated at the receiving UE. The inclusion of the serving cell ID field 404 may enable cross-carrier P-CSI report configuration selection (e.g., by indicating the target serving cell using the DCI received on the present serving cell, rather than waiting to use DCI on the target serving cell itself). Note that in cases where the serving cell ID field 404 is not used, the P-CSI ID field included in the DCI may be presumed to apply to the CC of the serving cell on which the DCI was itself received.

The other DCI fields 406 of the DCI format 400 may include other fields for the DCI format 400 beyond those related to the P-CSI report configuration (e.g., fields already present/used in DCI for format X_1 and/or X_2 as discussed above).

The cyclic redundancy check (CRC) bits 408 of the DCI format 400 may be applied at the UE to a radio network temporary identifier (RNTI) of the UE to allow the UE to identify the DCI format 400 as being for/directed to the UE.

FIG. 5 illustrates a group-common DCI format 500 for activating one or more P-CSI reporting configurations, according to an embodiment. The group-common DCI format 500 may enable activation/deactivation of P-CSI report configurations for multiple CCs and/or one or more UEs using those CCs in a single DCI.

The group-common DCI format 500 may include a plurality of CSI block fields (labelled as, e.g., “CSI block #1” through “CSI block #n” in FIG. 5). Each such CSI block field may include each of a P-CSI ID field that identifies a P-CSI report configuration and a serving cell ID field that identifies a serving cell corresponding to the CC to which the indicated P-CSI report configuration should apply.

One or more of the CSI block fields may be understood to be for a particular UE. For example, as illustrated, the first CSI block fields 506 (including the “CSI block #1” through the “CSI block #4”) of the group-common DCI format 500 are for a first UE 502, while additional CSI block fields 508 are instead intended for subsequent/other UE(s) 504.

Upon receiving the group-common DCI format 500, the UE may identify the location of one or more of its CSI block field(s) using a parameter “startingBitOfFormatX” provided by higher layer signaling from the base station to the UE.

Multiple CSI block fields for the same UE may be used in the case that the UE is configured with multiple CCs (e.g., under carrier aggregation (CA)). For example, it may be that the UE is configured with CC #1 through CC #4. In such a case, each of multiple CSI block fields for the UE included in the group-common DCI format may provide a P-CSI report configuration for a corresponding one of the CCs used by the UE. For example, in the group-common DCI format 500, the “CSI block #1” field may provide a P-CSI report configuration for CC #1 used by the first UE 502, the “CSI block #2” field may provide a P-CSI report configuration for CC #2 used by the first UE 502, the “CSI block #3” field provide a P-CSI report configuration for CC #3 used by the first UE 502, and the “CSI block #4” field may provide a P-CSI report configuration for CC #4 used by the first UE 502, in the manner illustrated.

The CRC bits 510 of the group-common DCI format 500 may be scrambled such that one or more UEs with an appropriate RNTI (e.g., a CSI-RNTI) can identify that the group-common DCI format 500 is for that UE and may thus contain one or more CSI block fields for that UE. The RNTI may be configured to the UE(s) by higher layer signaling from the base station.

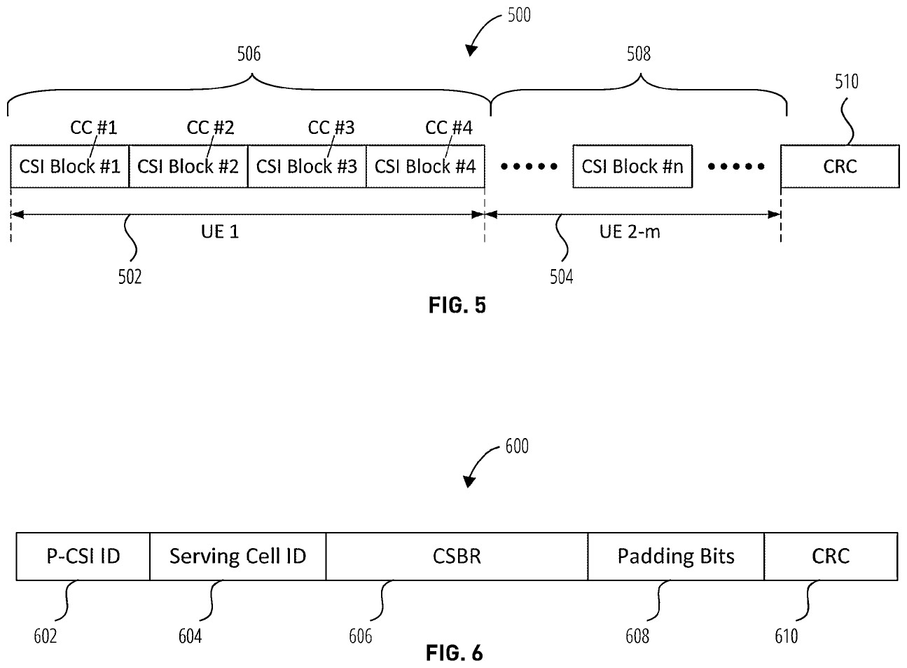

FIG. 6 illustrates a UE-specific DCI format 600 that may be used for P-CSI report activation, according to an embodiment. The UE-specific DCI format 600 may improve the reliability of DCI reception and/or allow for more flexible CSBR based on a latest beam report.

A P-CSI ID field 602 of the UE-specific DCI format 600 may indicate a particular P-CSI report configuration that should be activated at the receiving UE.

In at least some cases, the UE-specific DCI format 600 may include the serving cell ID field 604 as illustrated. A serving cell ID field 604 of the UE-specific DCI format 600 may indicate the serving cell corresponding to the CC for which the particular P-CSI report configuration should be activated at the receiving UE. The inclusion of the serving cell ID field 604 may enable cross-carrier P-CSI report configuration selection (e.g., by indicating the target serving cell using the DCI received on the present serving cell, rather than waiting to use DCI on the target serving cell itself). Note that in cases where the serving cell ID field is not used, the P-CSI ID field included in the DCI may be presumed to apply to the CC of the serving cell on which the DCI was itself received.

The UE-specific DCI format 600 may also include a CBSR field 606. In some cases, the CBSR field 606 uses a number of bits M that is equal to the size of the largest CSBR bitmap configuration from among a group of P-CSI reports configured to the UE. For example, in the group of P-CSI report configurations represented by index 1 in the table 100 of FIG. 1, CSBR bitmap configurations for 64, 32, 16, and eight bits are present, with the largest CSBR bitmap configuration being for 64 bits. Thus, in the case that this group of P-CSI report configurations is configured to the UE, the UE may understand that the CBSR field 606 uses 64 bits.

Then, once one P-CSI report configuration of the group is actively selected and is being used, it may be that a least significant number of bits P in the CBSR field 606 corresponding to the number of bits for the CSBR bitmap configuration for that particular P-CSI is used to indicate CSBR information between the base station and the UE.

The UE-specific DCI format 600 may also include the padding bits 608, which may be included such that the payload size of the UE-specific DCI format 600 equals the payload size of a DCI format already known to the wireless communications system. For example, in the case of an LTE or an NR wireless communications system, the padding bits 608 may be included in the UE-specific DCI format 600 such that it is the same size as a DCI format 0_1 or 1_1 that may be known in such systems. This may ensure that the use of the UE-specific DCI format 600 does not increase the amount of processing to perform blind decoding for DCI at the UE.

The UE-specific DCI format 600 may be identified by a dedicated RNTI (which may be called, e.g., a “Y-RNTI”) that is used to scramble the CRC bits 610 of the UE-specific DCI format 600, such that the UE is enabled to identify the UE-specific DCI format 600 using the dedicated RNTI.

It is further contemplated that additional flexibility could be achieved by incorporating the use of a group CSI report configurations (e.g., as a group of P-CSI report configurations as found in the table 100 as previously discussed) in the A-CSI case as well.

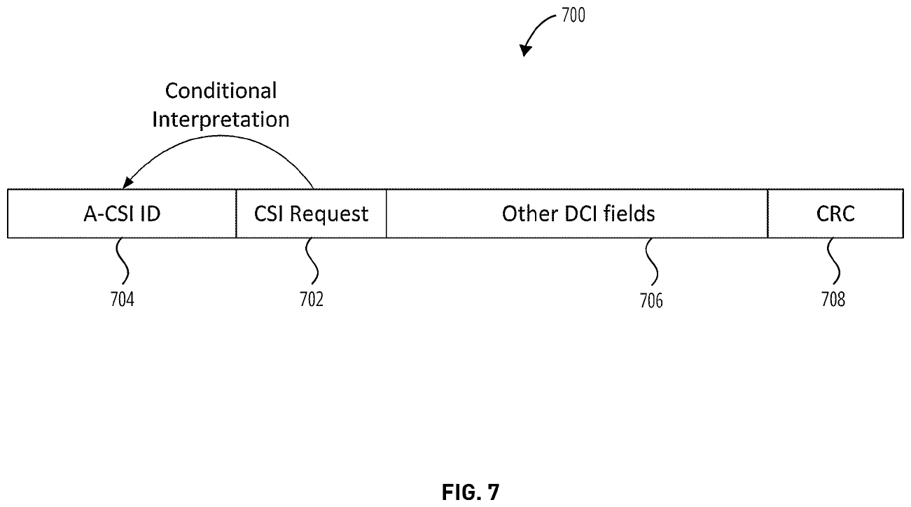

Discussion of FIG. 7 assumes the use of a group of A-CSI report configurations that matches, for example, one of the groups of P-CSI report configurations indexed in the table 100. Accordingly, while discussion will reference the table 100, it should be understood that this refers a corresponding table of groups of A-CSI configurations, one of which may be separately configured to the UE, or which may be a re-use by the UE of the group of P-CSI report configuration from the table 100 for the A-CSI case. In such a case, an individual configuration from the table 100 may be referred to as an A-CSI report configuration rather than a P-CSI report configuration.

It may be supposed that the UE is configured with the group of A-CSI report configurations corresponding to index 1 of the table 100. Further, the UE may be configured to select an A-CSI report configuration from this configured group based on a trigger state that is present in DCI.

FIG. 7 illustrates a DCI format 700 for triggering A-CSI, according to an embodiment. The DCI format 700 may include a CSI request field 702 for A-CSI that indicates a trigger state. It may be that, for example, this trigger state corresponds to or is associated with the group of A-CSI report configurations under discussion. Accordingly, in such a case, the UE will select an A-CSI report configuration from this group to generate the CSI report.

Further, as illustrated, the DCI format 700 includes an A-CSI ID field 704. As illustrated, the presence of both the CSI request field 702 and the A-CSI ID field 704 enables the UE to use conditional interpretation (based on the identification of this particular group of A-CSI report configurations from the use of the trigger state information of the CSI request field 702) to interpret that the value or index provided in the sub-configuration indicator A-CSI ID field 704 is for one of the sub-configurations of this group of A-CSI report configurations. Accordingly, the UE may use the indicated one of the group of A-CSI report configurations to generate an (aperiodic) CSI report to be sent to the base station.

The other DCI fields 706 of the DCI format 700 may include other fields for the DCI beyond those related to the sub-configuration (e.g., fields already present/used in DCI format X_1 and/or X_2 as may be used in some wireless communications systems, and where the DCI format 700 follows/is of one of those formats).

The CRC bits 708 of the DCI format 1400 may be applied at the UE to a RNTI of the UE to allow the UE to identify the DCI format 1400 as being for/directed to the UE.

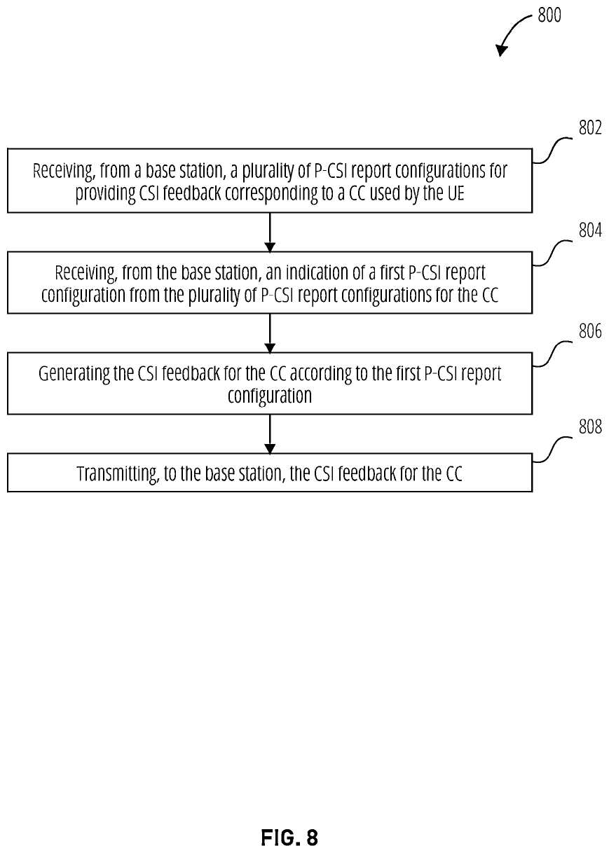

FIG. 8 illustrates a method 800 of a UE, according to an embodiment. The method 800 includes receiving 802, from a base station, a plurality of P-CSI report configurations for providing CSI feedback corresponding to a CC used by the UE.

The method 800 further includes receiving 804, from the base station, an indication of a first P-CSI report configuration from the plurality of P-CSI report configurations for the CC.

The method 800 further includes generating 806 the CSI feedback for the CC according to the first P-CSI report configuration.

The method 800 further includes transmitting 808, to the base station, the CSI feedback for the CC.

In some embodiments of the method 800, the plurality of P-CSI report configurations comprises a plurality of CSI-RS resource set configurations, wherein each of the plurality of CSI-RS resource set configurations is for a different number of CSI-RS ports for CSI-RS resources in a CSI-RS resource set.

In some embodiments of the method 800, the plurality of P-CSI report configurations comprises one or more of a plurality of antenna panel number configurations, a plurality of antenna array configurations, a plurality of CSBR bitmap configurations, a plurality of reporting periodicity configurations, and a plurality of frequency granularity configurations.

In some embodiments of the method 800, the indication of the first P-CSI report configuration is received in a MAC CE that includes a serving cell ID IE identifying a serving cell corresponding to the CC and a P-CSI ID IE identifying the first P-CSI report configuration. In some of these embodiments, the method 800 further includes applying the first P-CSI report configuration after a time instance n+T_{proc}, where n is one of a first slot of a PDSCH containing the MAC CE and a second slot of a PUCCH containing HARQ-ACK signaling for the PDSCH, and where T_{proc} is a processing time for applying the first P-CSI report configuration.

In some embodiments of the method 800, the indication of the first P-CSI report configuration is received in a scheduling DCI format comprising a P-CSI ID field identifying the first P-CSI report configuration. In some of these embodiments, the scheduling DCI format further comprises a serving cell ID field identifying a serving cell corresponding to the CC.

In some embodiments of the method 800, the indication of the first P-CSI report configuration is received in a group-common DCI format with CRC bits scrambled by a dedicated RNTI for a plurality of UEs that includes the UE, and a CSI block field of the UE in the group-common DCI format that corresponds to the CC identifies that the first sub-configuration is for the CC.

In some embodiments of the method 800, the indication of the first P-CSI report configuration is received in UE-specific DCI format corresponding to the UE that includes a serving cell ID field identifying a serving cell corresponding to the CC, a P-CSI field identifying the first P-CSI report configuration, and a CSBR field.

Embodiments contemplated herein include an apparatus comprising means to perform one or more elements of the method 800. This apparatus may be, for example, an apparatus of a UE (such as a wireless device 2402 that is a UE, as described herein).

Embodiments contemplated herein include one or more non-transitory computer-readable media comprising instructions to cause an electronic device, upon execution of the instructions by one or more processors of the electronic device, to perform one or more elements of the method 800. This non-transitory computer-readable media may be, for example, a memory of a UE (such as a memory 2406 of a wireless device 2402 that is a UE, as described herein).

Embodiments contemplated herein include an apparatus comprising logic, modules, or circuitry to perform one or more elements of the method 800. This apparatus may be, for example, an apparatus of a UE (such as a wireless device 2402 that is a UE, as described herein).

Embodiments contemplated herein include an apparatus comprising: one or more processors and one or more computer-readable media comprising instructions that, when executed by the one or more processors, cause the one or more processors to perform one or more elements of the method 800. This apparatus may be, for example, an apparatus of a UE (such as a wireless device 2402 that is a UE, as described herein).

Embodiments contemplated herein include a signal as described in or related to one or more elements of the method 800.

Embodiments contemplated herein include a computer program or computer program product comprising instructions, wherein execution of the program by a processor is to cause the processor to carry out one or more elements of the method 800. The processor may be a processor of a UE (such as a processor(s) 2404 of a wireless device 2402 that is a UE, as described herein). These instructions may be, for example, located in the processor and/or on a memory of the UE (such as a memory 2406 of a wireless device 2402 that is a UE, as described herein).

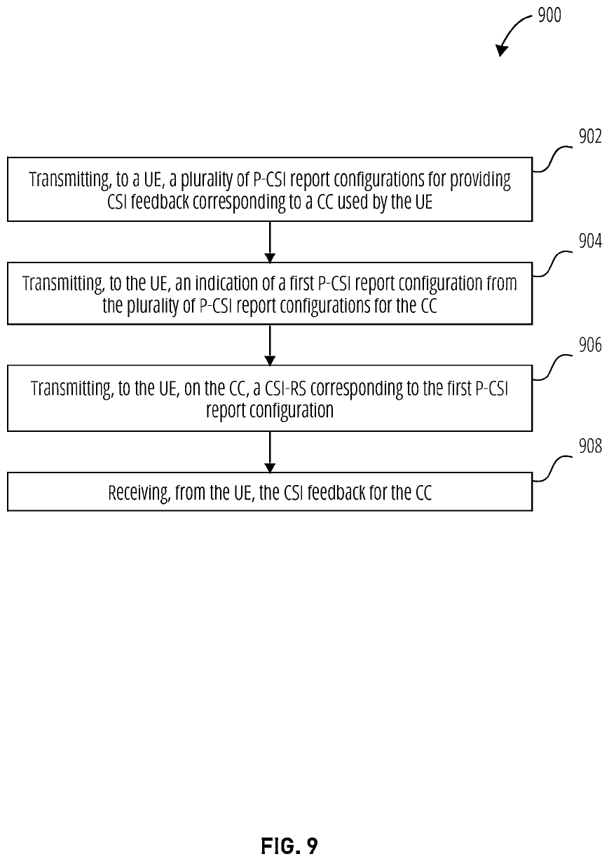

FIG. 9 illustrates a method 900 of a base station, according to an embodiment. The method 900 includes transmitting 902, to a UE, a plurality of P-CSI report configurations for providing CSI feedback corresponding to a CC used by the UE.

The method 900 further includes transmitting 904, to the UE, an indication of a first P-CSI report configuration from the plurality of P-CSI report configurations for the CC.

The method 900 further includes transmitting 906, to the UE, on the CC, a CSI-RS corresponding to the first P-CSI report configuration.

The method 900 further includes receiving 908, from the UE, the CSI feedback for the CC.

In some embodiments of the method 900, the plurality of P-CSI report configurations comprises a plurality of CSI-RS resource set configurations, wherein each of the plurality of CSI-RS resource set configurations is for a different number of CSI-RS ports for CSI-RS resources in a CSI-RS resource set.

In some embodiments of the method 900, the plurality of P-CSI report configurations comprises one or more of a plurality of antenna panel number configurations, a plurality of antenna array configurations, a plurality of CSBR bitmap configurations, a plurality of reporting periodicity configurations, and a plurality of frequency granularity configurations.

In some embodiments of the method 900, the indication of the first P-CSI report configuration is transmitted in a MAC CE that includes a serving cell ID IE identifying a serving cell corresponding to the CC and a P-CSI ID IE identifying the first P-CSI report configuration. In some of these embodiments, the method 900 further includes applying the first P-CSI report configuration after a time instance n+T_{proc}, where n is one of a first slot of a PDSCH containing the MAC CE and a second slot of a PUCCH containing HARQ-ACK signaling for the PDSCH, and where T_{proc} is a processing time for applying the first P-CSI report configuration.

In some embodiments of the method 900, the indication of the first P-CSI report configuration is transmitted in a scheduling DCI format comprising a P-CSI ID field identifying the first P-CSI report configuration. In some of these embodiments, the scheduling DCI format further comprises a serving cell ID field identifying a serving cell corresponding to the CC.

In some embodiments of the method 900, the indication of the first P-CSI report configuration is transmitted in a group-common DCI format with CRC bits scrambled by a dedicated RNTI for a plurality of UEs that includes the UE, and a CSI block field of the UE in the group-common DCI format that corresponds to the CC identifies that the first sub-configuration is for the CC.

In some embodiments of the method 900, the indication of the first P-CSI report configuration is transmitted in UE-specific DCI format corresponding to the UE that includes a serving cell ID field identifying a serving cell corresponding to the CC, a P-CSI field identifying the first P-CSI report configuration, and a CSBR field.

Embodiments contemplated herein include an apparatus comprising means to perform one or more elements of the method 900. This apparatus may be, for example, an apparatus of a base station (such as a network device 2418 that is a base station, as described herein).

Embodiments contemplated herein include one or more non-transitory computer-readable media comprising instructions to cause an electronic device, upon execution of the instructions by one or more processors of the electronic device, to perform one or more elements of the method 900. This non-transitory computer-readable media may be, for example, a memory of a base station (such as a memory 2422 of a network device 2418 that is a base station, as described herein).

Embodiments contemplated herein include an apparatus comprising logic, modules, or circuitry to perform one or more elements of the method 900. This apparatus may be, for example, an apparatus of a base station (such as a network device 2418 that is a base station, as described herein).

Embodiments contemplated herein include an apparatus comprising: one or more processors and one or more computer-readable media comprising instructions that, when executed by the one or more processors, cause the one or more processors to perform one or more elements of the method 900. This apparatus may be, for example, an apparatus of a base station (such as a network device 2418 that is a base station, as described herein).

Embodiments contemplated herein include a signal as described in or related to one or more elements of the method 900.

Embodiments contemplated herein include a computer program or computer program product comprising instructions, wherein execution of the program by a processing element is to cause the processing element to carry out one or more elements of the method 900. The processor may be a processor of a base station (such as a processor(s) 2420 of a network device 2418 that is a base station, as described herein). These instructions may be, for example, located in the processor and/or on a memory of the base station (such as a memory 2422 of a network device 2418 that is a base station, as described herein).

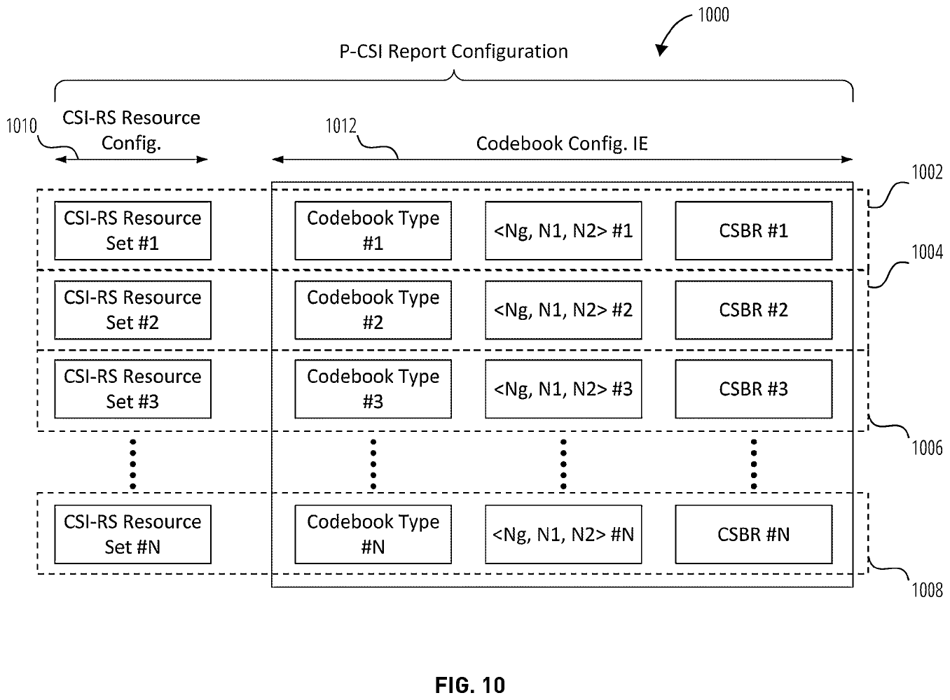

FIG. 10 illustrates a P-CSI report configuration 1000 that lists various sub-configurations for providing CSI feedback, according to an embodiment. The P-CSI report configuration 1000 includes a first sub-configuration 1002, a second sub-configuration 1004, a third sub-configuration 1006, and additional sub-configuration(s) up to and including the n^{th} sub-configuration 1008. Note that it is anticipated that in other embodiments, fewer than four sub-configurations may be present in a P-CSI report configuration. Per various embodiments of the P-CSI report configuration 1000 contemplated herein, it should be understood that there may be zero or more additional sub-configurations between the third sub-configuration 1006 and the n^{th} sub-configuration 1008.

As illustrated, each of the sub-configurations 1002 through 1008 includes a CSI-RS resource set configuration 1010. Each CSI-RS resource set configuration 1010 may be for a CSI-RS resource set that configures the UE for a use of a CSI-RS resource in the CSI-RS resource set having a different number of CSI-RS ports. For example, as illustrated, the first sub-configuration 1002 may use a “CSI-RS Resource Set #1” configuration that is for the use of a CSI-RS resource in the Set #1 having a number of CSI-RS ports A₁, the second sub-configuration 1004 may use a “CSI-RS Resource Set #2” configuration that is for the use of a CSI-RS resource in the Set #2 having a number of CSI-RS ports A₂, the third sub-configuration 1006 may use a “CSI-RS Resource Set #3” configuration that is for the use of a CSI-RS resource in the Set #3 having a number of CSI-RS ports A₃, and the n^{th} sub-configuration 1008 may use a “CSI-RS Resource Set #N” configuration that is for the use of a CSI-RS resource in the Set #N having a number of CSI-RS ports A_{N}, where A₁≠A₂≠A₃≠A_{N}.

Each of the sub-configurations 1002 through 1008 further includes a codebook configuration IE 1012. The codebook configuration IE 1012 may be define a codebook type (e.g., Type 1 or Type 2) that is to be used for CSI feedback per that sub-configuration.

The codebook configuration IE 1012 for each of the sub-configurations 1002 through 1008 may also include an <Ng, N1, N2> configuration, where Ng is the number of antenna panels, N1 indicates the number of antenna elements in horizontal direction and N2 indicates the number of antenna elements in vertical direction per that sub-configuration.

The codebook configuration IE 1012 for each of the sub-configurations 1002 through 1008 may also include a CSBR bitmap configuration (e.g., indicating a number of bits used in a CSBR bitmap for the corresponding sub-configuration).

Once a UE is configured with a P-CSI report configuration having multiple sub-configurations, a variety of methods for providing the UE with a signaling indication of a sub-configuration of the P-CSI report configuration to use are considered. In a first case, an indication of the P-CSI report configuration may be sent in a MAC CE. Such a MAC CE may be identified by a MAC subheader with a dedicated LCID for MAC CE that include such indications. The MAC CE may have a fixed size.

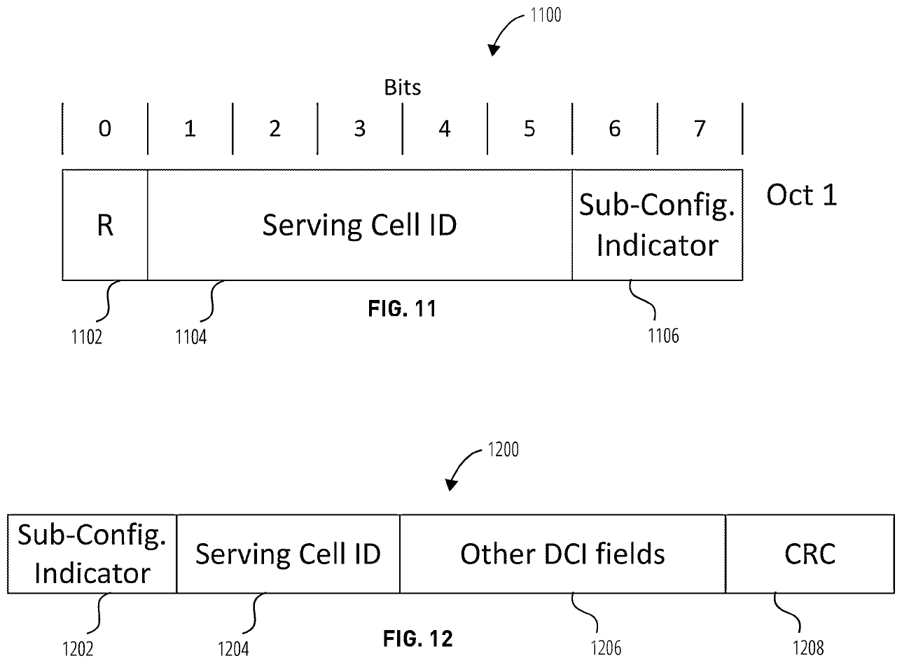

FIG. 11 illustrates a MAC CE 1100 for dynamically indicating a sub-configuration of a P-CSI report configuration to a UE, according to an embodiment. The MAC CE 1100 may include a number of octets N that together contain a pair of IEs. In the embodiment of FIG. 3, N=1 (other cases are contemplated). In the MAC CE 1100, there is one reserved bit 1102 (bit 0), five bits (bits 1 through 5) are used for a serving cell ID IE 1104 that indicates the identity of the serving cell for which this MAC CE is providing P-CSI report configuration indication, and two bits (bits 6 and 7) are used for a sub-configuration indicator IE 1106 to indicate the particular sub-configuration of the P-CSI report configuration that should be used on the CC of the identified serving cell.

The use of two bits for the sub-configuration indicator IE 1106 may correspond to the case where the number of sub-configurations in the P-CSI report configuration is four (where the four sub-configurations can be uniquely represented using two bits). More generally, it may be understood that a number of bits needed for a sub-configuration indicator IE 1106 may be log₂(M) when M sub-configurations are present in the P-CSI report configuration. To indicate a particular sub-configuration of the P-CSI report configuration, the sub-configuration indicator IE 1106 may indicate a value k that corresponds to the k^{th} sub-configuration. Accordingly, cases where many sub-configurations are present in the P-CSI report configuration, the MAC CE may use more than N=1 octets in order to accommodate the bits of an appropriately-sized sub-configuration indicator IE.

In some cases, the sub-configuration given by a MAC CE may be activated at a time instance n+T_{proc}. In some cases, n may be either a slot of a physical downlink shared channel (PDSCH) containing the MAC CE or a slot of a physical uplink control channel (PUCCH) containing hybrid automatic repeat request acknowledgement (HARQ-ACK) signaling for the PDSCH.

Further, T_{proc} may be a processing time for applying the first sub-configuration at the UE. In some cases, the value of T_{proc} may be pre-established by a specification that defines the behavior of the wireless communication system (e.g., the specification may define that T_{proc}=3 milliseconds (ms)). In some cases, it may be that such a specification provides a set of candidate values for T_{proc}, and that the UE then indicates an appropriate/desired T_{proc} (e.g., corresponding to a UE capability) to the base station via UE capability reporting/messaging.

In a second case of methods for providing the UE with a signaling indication of a sub-configuration of a P-CSI report configuration, a DCI format for activating/deactivating particular sub-configuration(s) from the P-CSI report configuration may be used.

FIG. 12 illustrates a DCI format 1200 for activating a sub-configuration of a P-CSI report configuration, according to an embodiment. The DCI format 1200 may represent, for example, a non-fallback scheduling DCI of a (modified) format of X_1 and or X_2, as may be understood in some specifications for some wireless communications systems.

A sub-configuration indicator 1202 of the DCI format 1200 may indicate a particular sub-configuration that should be activated at the receiving UE.

In at least some cases, the DCI format 1200 may include the serving cell ID field 1204 as illustrated. A serving cell ID field 1204 of the DCI format 1200 may indicate the serving cell corresponding to the CC for which the particular sub-configuration should be activated at the receiving UE. The inclusion of the serving cell ID field 1204 may enable cross-carrier sub-configuration selection (e.g., by indicating the target serving cell using the DCI received on the present serving cell, rather than waiting to use DCI on the target serving cell itself). Note that in cases where the serving cell ID field is not used, the sub-configuration indicator 1202 included in the DCI may be presumed to apply to the CC of the serving cell on which the DCI was itself received.

The other DCI fields 1206 of the DCI format 1200 may include other fields for the DCI beyond those related to the sub-configuration (e.g., fields already present/used in DCI for format X_1 and/or X_2 as discussed above).

The CRC bits 1208 of the DCI format 1200 may be applied at the UE to a RNTI of the UE to allow the UE to identify the DCI format 1200 as being for/directed to the UE.

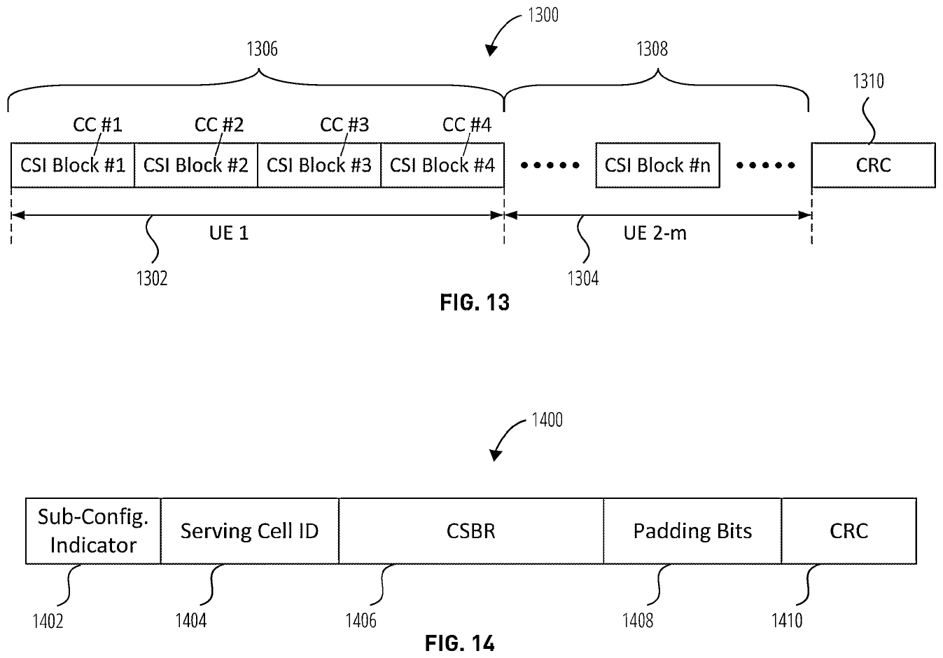

FIG. 13 illustrates a group-common DCI format 1300 for activating one or more sub-configurations of a P-CSI report configuration, according to an embodiment. The group-common DCI format 1300 may enable activation/deactivation of sub-configurations for multiple CCs and/or one or more UEs using those CCs in a single DCI.

The group-common DCI format 1300 may include a plurality of CSI block fields (labelled as, e.g., “CSI block #1” through “CSI block #n” in FIG. 13). Each such CSI block field may include each of a sub-configuration indicator field that identifies a sub-configuration and a serving cell ID field that identifies a serving cell corresponding to the CC to which the indicated sub-configuration should apply.

One or more of the CSI block fields may be understood to be for a particular UE. For example, as illustrated, the first CSI block fields 1306 (including the “CSI block #1” through the “CSI block #4”) of the group-common DCI format 1300 are for a first UE 1302, while additional CSI block fields 1308 are instead intended for subsequent/other UE(s) 1304.

Upon receiving the group-common DCI format 1300, the UE may identify the location of one or more of its CSI block field(s) using a parameter “startingBitOfFormatX” provided by higher layer signaling from the base station to the UE.

Multiple CSI block fields for the same UE may be used in the case that the UE is configured with multiple CCs (e.g., under CA). For example, it may be that the UE is configured with CC #1 through CC #4. In such a case, each of multiple CSI block fields for the UE included in the group-common DCI format may provide a sub-configuration for a corresponding one of the CCs used by the UE. For example, in the group-common DCI format 1300, the “CSI block #1” field may provide a sub-configuration for CC #1, the “CSI block #2” field may provide a sub-configuration for CC #2, the “CSI block #3” field provide a sub-configuration for CC #3, and the “CSI block #4” field may provide a sub-configuration for CC #4, in the manner illustrated.

The CRC bits 1310 of the group-common DCI format 1300 may be scrambled such that one or more UEs with an appropriate RNTI (e.g., a CRC-RNTI) can identify that the group-common DCI format 1300 is for that UE and may thus contain one or more CSI block fields for that UE. The RNTI may be configured to the UE(s) by higher layer signaling from the base station.

FIG. 14 illustrates a UE-specific DCI format 1400 that may be used for activation of a sub-configuration of a P-CSI report configuration, according to an embodiment. The UE-specific DCI format 1400 may improve the reliability of DCI reception and/or allow for more flexible CSBR based on a latest beam report.

A sub-configuration indicator field 1402 of the UE-specific DCI format 1400 may indicate a particular sub-configuration that should be activated at the receiving UE.

In at least some cases, the UE-specific DCI format 1400 may include the serving cell ID field 1404 as illustrated. A serving cell ID field 1404 of the UE-specific DCI format 1400 may indicate the serving cell corresponding to the CC for which the particular sub-configuration should be activated at the receiving UE. The inclusion of the serving cell ID field 1404 may enable cross-carrier sub-configuration selection (e.g., by indicating the target serving cell using the DCI received on the present serving cell, rather than waiting to use DCI on the target serving cell itself). Note that in cases where the serving cell ID field is not used, the sub-configuration indicator field 1402 included in the DCI may be presumed to apply to the CC of the serving cell on which the DCI was itself received.

The UE-specific DCI format 1400 may also include a CBSR field 1406. In some cases, the CBSR field 1406 uses a number of bits M that is equal to the size of the largest CSBR bitmap configuration from among the sub-configurations of the P-CSI report configuration. For example, with reference to the P-CSI report configuration 1000 of FIG. 10, it may be that the first sub-configuration 1002 uses a first CSBR bitmap configuration (“CSBR #1”) for 64 bits, that the second sub-configuration 1004 uses a second CSBR bitmap configuration (“CSBR #2”) for 32 bits, the CSI-RS resource set configuration 1010 uses a third CSBR bitmap configuration (“CSBR #3”) for 16 bits, and the n^{th} sub-configuration 1008 uses a fourth CSBR bitmap configuration (“CSBR #4”) for eight bits (and this case may suppose no other sub-configurations in the P-CSI report configuration 1000). In such a case, the sub-configuration with the largest CSBR bitmap configuration is 64 bits. Thus, the UE may understand that the CBSR field 1406 uses 64 bits.

Then, once one sub-configuration is actively selected and is being used, it may be that a least significant number of bits P in the CBSR field 1406 corresponding to the number of bits for the CSBR bitmap configuration for that particular sub-configuration is used to indicate CSBR information between the base station and the UE.

The UE-specific DCI format 1400 may also include the padding bits 1408, which may be included such that the payload size of the UE-specific DCI format 1400 equals the payload size of a DCI format already known to the wireless communications system. For example, in the case of an LTE or an NR wireless communications system, the padding bits 1408 may be included in the UE-specific DCI format 1400 such that it is the same size as a DCI format 0_1 or 1_1 that may be known in such systems. This may ensure that the use of the UE-specific DCI format 1400 does not increase the amount of processing to perform blind decoding for DCI at the UE.

The UE-specific DCI format 1400 may be identified by a dedicated RNTI (which may be called, e.g., a “Y-RNTI”) that is used to scramble the CRC bits 1410 of the UE-specific DCI format 1400, such that the UE is enabled to identify the UE-specific DCI format 1400 using the dedicated RNTI.

In some wireless communications systems, it may be that if a resource setting linked to a “CSI-ReportConfig” has multiple aperiodic CSI-RS resource sets, only one of these aperiodic CSI-RS resources sets is selected and associated with an aperiodic CSI (A-CSI) trigger state. This selection may be performed by RRC signaling.

However, it is contemplated that additional flexibility could be achieved by incorporating the use of CSI report configurations having multiple sub-configurations (e.g., such as the P-CSI report configuration 1000 previously discussed) in the A-CSI case as well.

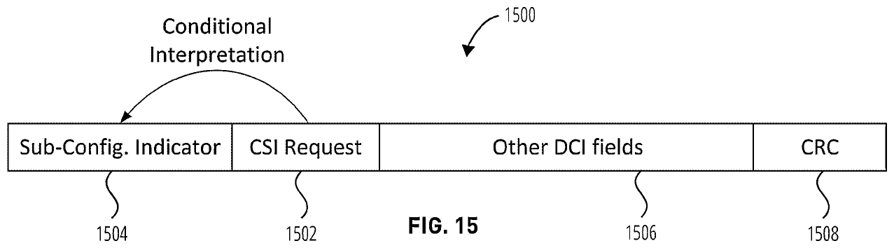

Discussion of FIG. 15 assumes the use of an A-CSI report configuration that matches the P-CSI report configuration 1000. Accordingly, while discussion will reference elements of the P-CSI report configuration 1000, it should be understood that this refers to a corresponding A-CSI configuration, which may be separately configured to the UE, or which may be a re-use by the UE of the P-CSI report configuration 1000 for the A-CSI case.

Accordingly, this A-CSI report configuration may be one of multiple A-CSI report configurations that may be used by the UE, and may be selected for use from among the multiple A-CSI report configurations according to a trigger state that is present in DCI.

FIG. 15 illustrates a DCI format 1500 for triggering A-CSI, according to an embodiment. The DCI format 1500 may include a CSI request field 1502 for A-CSI that indicates a trigger state. It may be that, for example, this trigger state corresponds to or is associated with the A-CSI report configuration matching the P-CSI report configuration 1000 of FIG. 10. Accordingly, in such a case, the UE will use this particular A-CSI report configuration to generate the CSI report (as opposed to another A-CSI report configuration that may exist).

Further, as illustrated, the DCI format 1500 includes a sub-configuration indicator field 1504. As illustrated, the presence of both the CSI request field 1502 and the sub-configuration indicator field 1504 enables the UE to use conditional interpretation (based on the identification of this particular A-CSI report configuration from the use of the trigger state information of the CSI request field 1502) to interpret that the value or index provided in the sub-configuration indicator field 1504 is for one of the sub-configurations 1002 through 1008 of this A-CSI report configuration. Accordingly, the UE may use the indicated one of the 1002 through 1008 to generate an (aperiodic) CSI report to be sent to the base station.

The other DCI fields 1506 of the DCI format 1500 may include other fields for the DCI beyond those related to the sub-configuration (e.g., fields already present/used in DCI format X_1 and/or X_2 as may be used in some wireless communications systems, and where the DCI format 1500 follows/is of one of those formats).

The CRC bits 1508 of the DCI format 1500 may be applied at the UE to a RNTI of the UE to allow the UE to identify the DCI format 1500 as being for/directed to the UE.

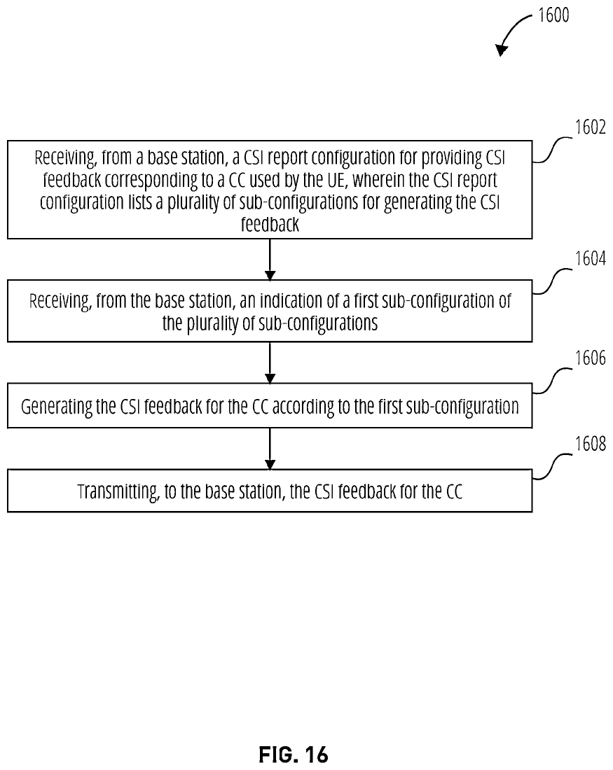

FIG. 16 illustrates a method 1600 of a UE, according to an embodiment. The method 1600 includes receiving 1602, from a base station, a CSI report configuration for providing CSI feedback corresponding to a CC used by the UE, wherein the CSI report configuration lists a plurality of sub-configurations for generating the CSI feedback.

The method 1600 further includes receiving 1604, from the base station, an indication of a first sub-configuration of the plurality of sub-configurations.

The method 1600 further includes generating 1606 the CSI feedback for the CC according to the first sub-configuration.

The method 1600 further includes transmitting 1608, to the base station, the CSI feedback for the CC.

In some embodiments of the method 1600, the plurality of sub-configurations comprise a plurality of CSI-RS resource set configurations, wherein each of the plurality of CSI-RS resource set configurations is for a different number of CSI-RS ports for CSI-RS resources in a CSI-RS resource set.

In some embodiments of the method 1600, the plurality of sub-configurations comprises a plurality of codebook configurations. In some of these embodiments, the plurality of codebook configurations comprises one or more of a plurality of codebook types, a plurality of antenna panel number configurations, a plurality of antenna array configurations, and a plurality of CSBR bitmap configurations.

In some embodiments of the method 1600, the indication of the first sub-configuration is received in a MAC CE that includes a serving cell ID IE identifying a serving cell corresponding to the CC and a sub-configuration indicator IE identifying the first sub-configuration. In some of these embodiments, the method 1600 further includes applying the first sub-configuration after a time n+T_{proc}, where n is one of a first slot of a PDSCH containing the MAC CE and a second slot of a PUCCH containing HARQ-ACK signaling for the PDSCH, and where T_{proc} is a processing time for applying the first sub-configuration.

In some embodiments of the method 1600, the indication of the first sub-configuration is received in scheduling DCI format that includes a sub-configuration indicator field identifying the first sub-configuration. In some of these embodiments, the scheduling DCI format further comprises a serving cell ID field identifying a serving cell corresponding to the CC.

In some embodiments of the method 1600, the indication of the first sub-configuration is received in a group-common DCI format with CRC bits scrambled by a dedicated RNTI for a plurality of UEs that includes the UE, and a CSI block field of the UE in the group-common DCI format that corresponds to the CC identifies that the first sub-configuration is for the CC.

In some embodiments of the method 1600, the indication of the first sub-configuration is received in UE-specific DCI format corresponding to the UE that includes a serving cell ID field identifying a serving cell corresponding to the CC, a sub-configuration indicator field identifying the first sub-configuration, and a CSBR field.

In some embodiments of the method 1600, the indication of the first sub-configuration is received in an A-CSI report triggering DCI for the CSI feedback that includes a sub-configuration indicator field identifying the first sub-configuration.

Embodiments contemplated herein include an apparatus comprising means to perform one or more elements of the method 1600. This apparatus may be, for example, an apparatus of a UE (such as a wireless device 2402 that is a UE, as described herein).

Embodiments contemplated herein include one or more non-transitory computer-readable media comprising instructions to cause an electronic device, upon execution of the instructions by one or more processors of the electronic device, to perform one or more elements of the method 1600. This non-transitory computer-readable media may be, for example, a memory of a UE (such as a memory 2406 of a wireless device 2402 that is a UE, as described herein).

Embodiments contemplated herein include an apparatus comprising logic, modules, or circuitry to perform one or more elements of the method 1600. This apparatus may be, for example, an apparatus of a UE (such as a wireless device 2402 that is a UE, as described herein).

Embodiments contemplated herein include an apparatus comprising: one or more processors and one or more computer-readable media comprising instructions that, when executed by the one or more processors, cause the one or more processors to perform one or more elements of the method 1600. This apparatus may be, for example, an apparatus of a UE (such as a wireless device 2402 that is a UE, as described herein).

Embodiments contemplated herein include a signal as described in or related to one or more elements of the method 1600.

Embodiments contemplated herein include a computer program or computer program product comprising instructions, wherein execution of the program by a processor is to cause the processor to carry out one or more elements of the method 1600. The processor may be a processor of a UE (such as a processor(s) 2404 of a wireless device 2402 that is a UE, as described herein). These instructions may be, for example, located in the processor and/or on a memory of the UE (such as a memory 2406 of a wireless device 2402 that is a UE, as described herein).

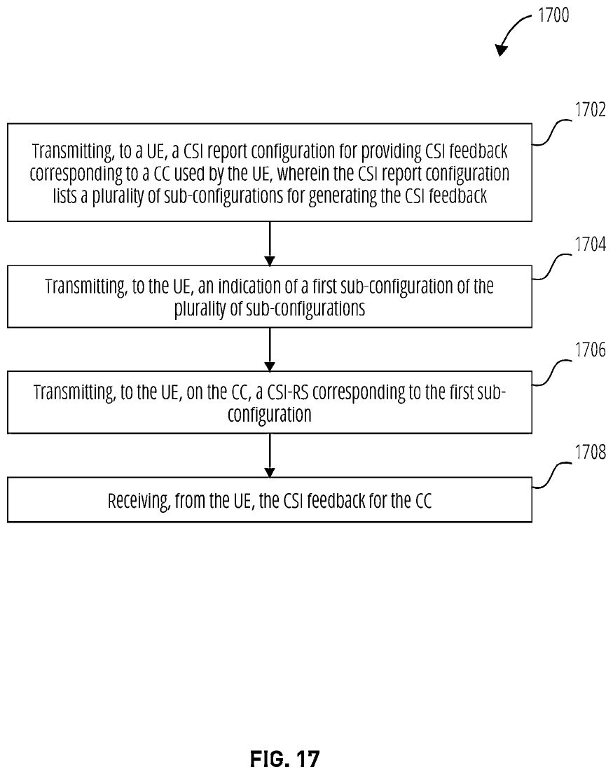

FIG. 17 illustrates a method 1700 of a base station, according to an embodiment. The method 1700 includes transmitting 1702, to a UE, a CSI report configuration for providing CSI feedback corresponding to a CC used by the UE, wherein the CSI report configuration lists a plurality of sub-configurations for generating the CSI feedback.

The method 1700 further includes transmitting 1704, to the UE, an indication of a first sub-configuration of the plurality of sub-configurations.

The method 1700 further includes transmitting 1706, to the UE, on the CC, a CSI-RS corresponding to the first sub-configuration.

The method 1700 further includes receiving 1708, from the UE, the CSI feedback for the CC.

In some embodiments of the method 1700, the plurality of sub-configurations comprise a plurality of CSI-RS resource set configurations, wherein each of the plurality of CSI-RS resource set configurations is for a different number of CSI-RS ports for CSI-RS resources in a CSI-RS resource set.

In some embodiments of the method 1700, the plurality of sub-configurations comprises a plurality of codebook configurations. In some of these embodiments, the plurality of codebook configurations comprises one or more of a plurality of codebook types, a plurality of antenna panel number configurations, a plurality of antenna array configurations, and a plurality of CSBR bitmap configurations.

In some embodiments of the method 1700, the indication of the first sub-configuration is transmitted in a MAC CE that includes a serving cell ID IE 1104 ID IE identifying a serving cell corresponding to the CC and a sub-configuration indicator IE identifying the first sub-configuration. In some of these embodiments, the method 1700 further includes applying the first sub-configuration after a time n+T_{proc}, where n is one of a first slot of a PDSCH containing the MAC CE and a second slot of a PUCCH containing HARQ-ACK signaling for the PDSCH, and where T_{proc} is a processing time for applying the first sub-configuration.

In some embodiments of the method 1700, the indication of the first sub-configuration is transmitted in scheduling DCI format that includes a sub-configuration indicator field identifying the first sub-configuration. In some of these embodiments, the scheduling DCI format further comprises a serving cell ID field identifying a serving cell corresponding to the CC.

In some embodiments of the method 1700, the indication of the first sub-configuration is transmitted in a group-common DCI format with CRC bits scrambled by a dedicated RNTI for a plurality of UEs that includes the UE, and a CSI block field of the UE in the group-common DCI format that corresponds to the CC identifies that the first sub-configuration is for the CC.

In some embodiments of the method 1700, the indication of the first sub-configuration is transmitted in UE-specific DCI format corresponding to the UE that includes a serving cell ID field identifying a serving cell corresponding to the CC, a sub-configuration indicator field identifying the first sub-configuration, and a CSBR field.

In some embodiments of the method 1700, the indication of the first sub-configuration is transmitted in an A-CSI report triggering DCI for the CSI feedback that includes a sub-configuration indicator field identifying the first sub-configuration.

Embodiments contemplated herein include an apparatus comprising means to perform one or more elements of the method 1700. This apparatus may be, for example, an apparatus of a base station (such as a network device 2418 that is a base station, as described herein).

Embodiments contemplated herein include one or more non-transitory computer-readable media comprising instructions to cause an electronic device, upon execution of the instructions by one or more processors of the electronic device, to perform one or more elements of the method 1700. This non-transitory computer-readable media may be, for example, a memory of a base station (such as a memory 2422 of a network device 2418 that is a base station, as described herein).

Embodiments contemplated herein include an apparatus comprising logic, modules, or circuitry to perform one or more elements of the method 1700. This apparatus may be, for example, an apparatus of a base station (such as a network device 2418 that is a base station, as described herein).

Embodiments contemplated herein include an apparatus comprising: one or more processors and one or more computer-readable media comprising instructions that, when executed by the one or more processors, cause the one or more processors to perform one or more elements of the method 1700. This apparatus may be, for example, an apparatus of a base station (such as a network device 2418 that is a base station, as described herein).

Embodiments contemplated herein include a signal as described in or related to one or more elements of the method 1700.

Embodiments contemplated herein include a computer program or computer program product comprising instructions, wherein execution of the program by a processing element is to cause the processing element to carry out one or more elements of the method 1700. The processor may be a processor of a base station (such as a processor(s) 2420 of a network device 2418 that is a base station, as described herein). These instructions may be, for example, located in the processor and/or on a memory of the base station (such as a memory 2422 of a network device 2418 that is a base station, as described herein).

It may be that in some cases, the use of a non-uniform linear array (NULA) arrangement for a CSI feedback process may provide better performance than the case of using a uniform linear array (ULA) arrangement. For example, beamforming performance may be improved in the case of an NULA arrangement (e.g., be helping to alleviate beam leakage and/or cross-beam interference).

Accordingly, a bitmap indicator may be used to indicate a 2-D sparse array pattern that is used with a configurable/arbitrary number and location of CSI-RS ports (e.g., as reduced over a base case of using all possible CSI-RS antenna ports, in order to reduce network power usage).

The base station may provide the UE with a CSI-RS port number configuration identifying a number M of CSI-RS ports. The base station may also provide the UE with a panel array configuration for a panel array for an M-port CSI-RS (e.g., where M=2*N1*N2, with N1 indicating the number of antenna elements in horizontal direction and N2 indicating the number of antenna elements in vertical direction). These items of information may be provided as previously elsewhere herein (e.g., as part of the indication of one of a plurality of a P-CSI configurations for the UE to use, or as part of the indication of a sub-configuration of a CSI configuration (e.g., for P-CSI and/or A-CSI) for the UE to use).

Then, the base station may provide the UE with an M-bit CSI-RS ports bitmap field. Each bit of this field may correspond to a CSI-RS port. These bits may be arranged according to a correspondence of the bits to CSI-RS port indexes that they represent (e.g., the bits may be arranged in increasing order of CSI-RS port index). It may be that when a given bit i is set to 0 within this bitmap, this indicates to the UE that the corresponding CSI-RS port (of index i) is off during the associated CSI-RS transmission from the base station. Further, it may be that when a given bit i is set to 1 within this bitmap, this indicates to the UE that the corresponding CSI-RS port (of index i) is on during the associated CSI-RS transmission from the base station. Accordingly, it may be said that an M-bit CSI-RS ports bitmap field provides a CSI-RS port configuration for the UE.

This M-bit CSI-RS ports bitmap field may be conveyed from the base station to the UE in a number of possible ways. In some cases, the M-bit CSI-RS ports bitmap field may be conveyed using a MAC CE that is identified based on a dedicated LCID is for the communication of such an M-bit CSI-RS ports bitmap field. The MAC CE may also include a serving cell ID IE that indicates the identity of the serving cell to which the M-bit CSI-RS ports bitmap applies.

In some cases, the M-bit CSI-RS ports bitmap field may be conveyed using a unicast scheduling DCI format for the UE that incorporates this field. Accordingly, the M-bit CSI-RS ports bitmap is applied for the serving cell addressed by the scheduling DCI format. Note that it is contemplated that this field could be added to one or more DCI formats that are known to a wireless communications system.

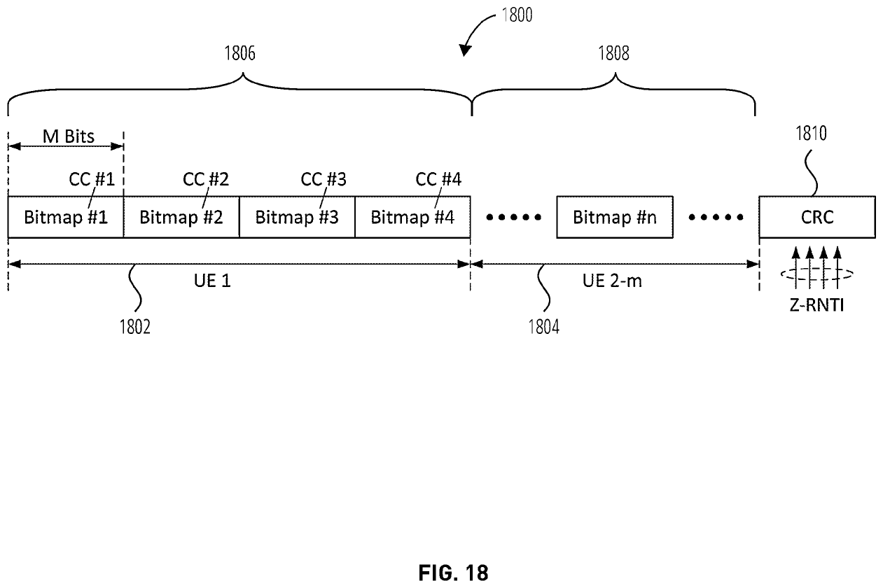

FIG. 18 illustrates a group-common DCI format 1800 for communicating one or more M-bit CSI-RS ports bitmap fields, according to an embodiment. The group-common DCI format 1800 may enable group-wise communication of M-bit CSI-RS ports bitmap fields for multiple CCs and/or one or more UEs using those CCs in a single DCI.

The group-common DCI format 1800 may include a plurality of M-bit CSI-RS ports bitmap fields (labelled as, e.g., “Bitmap #1” through “Bitmap #n” in FIG. 5). Each such M-bit CSI-RS ports bitmap field may include a CSI-RS ports bitmap.

One or more of the M-bit CSI-RS ports bitmap fields may be understood to be for a particular UE. For example, as illustrated, the first M-bit CSI-RS ports bitmap fields 1806 (including the “Bitmap #1” through the “Bitmap #4”) of the group-common DCI format 1800 are for a first UE 1802, while additional M-bit CSI-RS ports bitmap fields 1808 are instead intended for subsequent/other UE(s) 1804.

For a given UE, the location of an M-bit CSI-RS ports bitmap field for a serving cell/CC used by the UE is first provided by higher layer signaling from the base station to the UE using a parameter “startingBitOfFormatX.” Upon receiving the group-common DCI format 1800, the UE may identify the corresponding location of one or more of its M-bit CSI-RS ports bitmap field(s) for different serving cells/CCs based on the “startingBitOfFormatX” for that particular serving cell/CC.

Multiple M-bit CSI-RS ports bitmap fields for the same UE may be used in the case that the UE is configured with multiple CCs (e.g., under a CA procedure). For example, it may be that the UE is configured with CC #1 through CC #4. In such a case, each of multiple M-bit CSI-RS ports bitmap fields for the UE included in the group-common DCI format may be for one of the CCs used by the UE. For example, in the group-common DCI format 1800, the “Bitmap #1” field may provide a CSI-RS port configuration for CC #1, the “Bitmap #2” field may provide a CSI-RS port configuration for CC #2, the “Bitmap #3” field may provide a CSI-RS port configuration for CC #3, and the “Bitmap #4” field may provide a CSI-RS port configuration for CC #4, in the manner illustrated.

Note that each of the M-bit CSI-RS ports bitmap fields may be of different sizes, to correspond to the number M of CSI-RS ports configured for that particular CC at that particular UE.

The CRC bits 1810 of the group-common DCI format 1800 may be scrambled such that one or more UEs with an appropriate RNTI (e.g., a Z-RNTI) can identify that the group-common DCI format 1800 is for that UE and may thus contain one or more M-bit CSI-RS ports bitmap fields for that UE. The RNTI may be configured to the UE(s) by higher layer signaling from the base station.

Once an M-bit CSI-RS ports bitmap field (e.g., for a particular CC) has been conveyed to the UE, it may be used at the UE for deriving a channel measurement of a CSI parameter (e.g., a rank indicator (RI), a precoding matrix indicator (PMI), and/or a channel quality indicator (CQI), etc.). In such a case, the UE may set the channel response for the CSI-RS ports having i=0 in the M-bit CSI-RS ports bitmap field to zero (e.g., the UE does not use these CSI-RS ports when performing the channel measurement). Accordingly, the UE will effectively consider only the CSI-RS ports having i=1 in the M-bit CSI-RS ports bitmap when performing the channel measurement.

Note that the UE may still use an M-port codebook for computing the measurement of the received CSI-RS. By using the M-port codebook (e.g., as opposed to using a reduced codebook that matches the number of bits i=1 in the CSI-RS ports bitmap field), it may be that beam leakage and/or cross beam interference issues may be reduced.

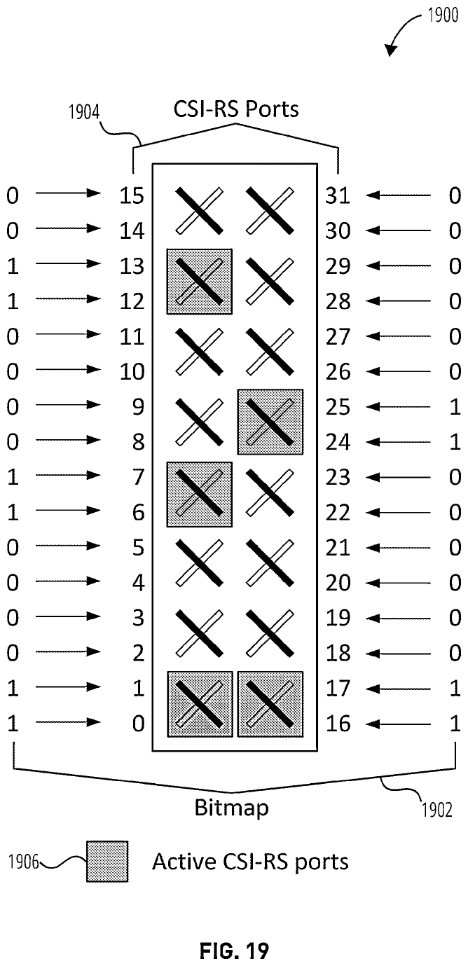

FIG. 19 illustrates an antenna panel 1900 non-uniform linear array (NULA) arrangement for a CSI feedback process, according to an embodiment. The antenna panel 1900 uses M=32, with N1=8 and N2=2 (and note that the view of the antenna panel 1900 in FIG. 19 is rotated 90 degrees).

The antenna panel 1900 corresponds to the use of a 2D sparse array that represents a parallel co-prime array (PCA) arrangement. An M-bit CSI-RS ports bitmap 1902 (where M=32) of <12000011000011001100000011000000> has been signaled to the UE and applied to the CSI-RS ports 1904 according to their increasing indexes, as illustrated. Accordingly, the antenna panel 1900 uses the active CSI-RS ports 1906 as indicated for the CSI-RS transmission to the UE, and the UE considers the active CSI-RS ports 1906 when performing channel measurement.

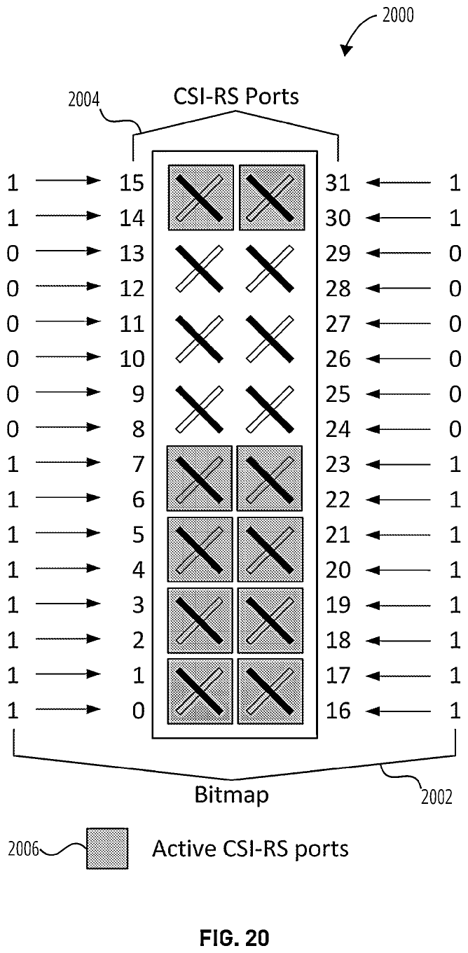

FIG. 20 illustrates an antenna panel 2000 non-uniform linear array (NULA) arrangement for a CSI feedback process, according to an embodiment. The antenna panel 2000 uses M=32, with N1=8 and N2=2 (and note that the view of the antenna panel 2000 in FIG. 20 is rotated 90 degrees).

The antenna panel 2000 corresponds to the use of a 2D sparse array that represents a parallel nested array (PNA) arrangement. An M-bit CSI-RS ports bitmap 2002 (where M=32) of <11111111000000111111111100000011> has been signaled to the UE and applied to the CSI-RS ports 2004 according to their increasing indexes, as illustrated. Accordingly, the antenna panel 2000 uses the active CSI-RS ports 2006 as indicated for the CSI-RS to the UE, and the UE considers the active CSI-RS ports 2006 when performing channel measurement.

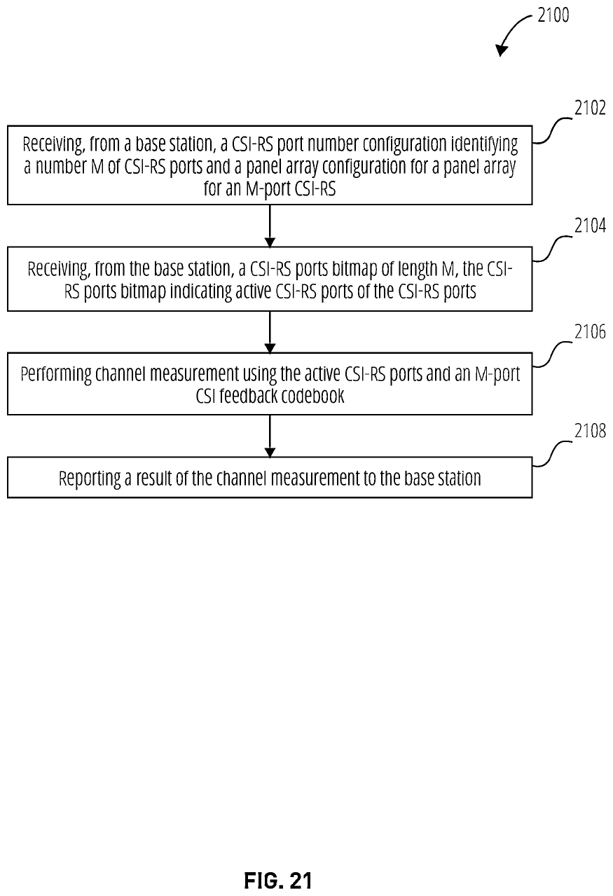

FIG. 21 illustrates a method 2100 of a UE, according to an embodiment. The method 2100 includes receiving 2102, from a base station, a CSI-RS port number configuration identifying a number M of CSI-RS ports and a panel array configuration for a panel array for an M-port CSI-RS.

The method 2100 further includes receiving 2104, from the base station, a CSI-RS ports bitmap of length M, the CSI-RS ports bitmap indicating active CSI-RS ports of the CSI-RS ports.

The method 2100 further includes performing 2106 channel measurement using the active CSI-RS ports and an M-port CSI feedback codebook.

The method 2100 further includes reporting 2108 a result of the channel measurement to the base station.

In some embodiments of the method 2100, the channel measurement comprises computing one of an RI, a PMI, and a CQI.

In some embodiments of the method 2100, the active CSI-RS ports correspond to one or more of a PNA configuration relative to the panel array and a PCA configuration relative to the panel array.

In some embodiments of the method 2100, the CSI-RS ports bitmap is received in a MAC CE identified by a dedicated LCID indicating that the MAC CE contains the CSI-RS ports bitmap.

In some embodiments of the method 2100, the CSI-RS ports bitmap is received in a unicast scheduling DCI format for the UE.

In some embodiments of the method 2100, the CSI-RS ports bitmap is received in a group-common DCI format for a plurality of UEs that includes the UE. In some of these embodiments, the CSI-RS ports bitmap is one of a plurality of CSI-RS ports bitmaps used by the UE in the group-common DCI format, wherein the plurality of CSI-RS ports bitmaps correspond to a plurality of CCs used by the UE.

Embodiments contemplated herein include an apparatus comprising means to perform one or more elements of the method 2100. This apparatus may be, for example, an apparatus of a UE (such as a wireless device 2402 that is a UE, as described herein).

Embodiments contemplated herein include one or more non-transitory computer-readable media comprising instructions to cause an electronic device, upon execution of the instructions by one or more processors of the electronic device, to perform one or more elements of the method 2100. This non-transitory computer-readable media may be, for example, a memory of a UE (such as a memory 2406 of a wireless device 2402 that is a UE, as described herein).

Embodiments contemplated herein include an apparatus comprising logic, modules, or circuitry to perform one or more elements of the method 2100. This apparatus may be, for example, an apparatus of a UE (such as a wireless device 2402 that is a UE, as described herein).

Embodiments contemplated herein include an apparatus comprising: one or more processors and one or more computer-readable media comprising instructions that, when executed by the one or more processors, cause the one or more processors to perform one or more elements of the method 2100. This apparatus may be, for example, an apparatus of a UE (such as a wireless device 2402 that is a UE, as described herein).

Embodiments contemplated herein include a signal as described in or related to one or more elements of the method 2100.

Embodiments contemplated herein include a computer program or computer program product comprising instructions, wherein execution of the program by a processor is to cause the processor to carry out one or more elements of the method 2100. The processor may be a processor of a UE (such as a processor(s) 2404 of a wireless device 2402 that is a UE, as described herein). These instructions may be, for example, located in the processor and/or on a memory of the UE (such as a memory 2406 of a wireless device 2402 that is a UE, as described herein).

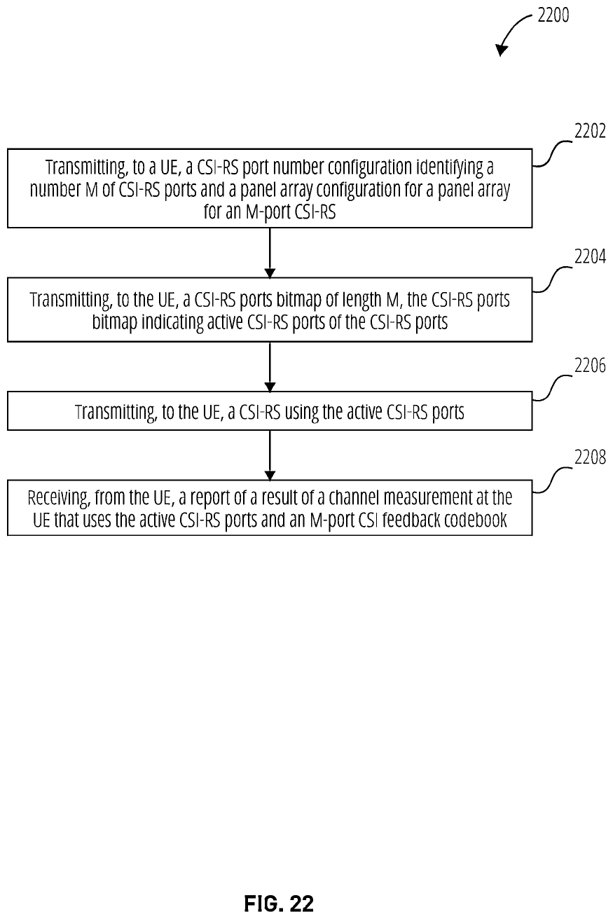

FIG. 22 illustrates a method 2200 of a base station, according to an embodiment. The method 2200 includes transmitting 2202, to a UE, a CSI-RS port number configuration identifying a number M of CSI-RS ports and a panel array configuration for a panel array for an M-port CSI-RS.

The method 2200 further includes transmitting 2204, to the UE, a CSI-RS ports bitmap of length M, the CSI-RS ports bitmap indicating active CSI-RS ports of the CSI-RS ports.

The method 2200 further includes transmitting 2206 to the UE, a CSI-RS using the active CSI-RS ports.

The method 2200 further includes receiving 2208, from the UE, a report of a result of a channel measurement at the UE that uses the active CSI-RS ports and an M-port CSI feedback codebook.

In some embodiments of the method 2200, the channel measurement comprises computing one of an RI, a PMI, and a CQI.

In some embodiments of the method 2200, the active CSI-RS ports correspond to one or more of a PNA configuration relative to the panel array and a PCA configuration relative to the panel array.

In some embodiments of the method 2200, the CSI-RS ports bitmap is transmitted in a MAC CE identified by a dedicated LCID indicating that the MAC CE contains the CSI-RS ports bitmap.

In some embodiments of the method 2200, the CSI-RS ports bitmap is transmitted in a unicast scheduling DCI format for the UE.

In some embodiments of the method 2200, the CSI-RS ports bitmap is transmitted in a group-common DCI format for a plurality of UEs that includes the UE. In some of these embodiments, the CSI-RS ports bitmap is one of a plurality of CSI-RS ports bitmaps used by the UE in the group-common DCI format, wherein the plurality of CSI-RS ports bitmaps correspond to a plurality of CCs used by the UE.

Embodiments contemplated herein include an apparatus comprising means to perform one or more elements of the method 2200. This apparatus may be, for example, an apparatus of a base station (such as a network device 2418 that is a base station, as described herein).

Embodiments contemplated herein include one or more non-transitory computer-readable media comprising instructions to cause an electronic device, upon execution of the instructions by one or more processors of the electronic device, to perform one or more elements of the method 2200. This non-transitory computer-readable media may be, for example, a memory of a base station (such as a memory 2422 of a network device 2418 that is a base station, as described herein).

Embodiments contemplated herein include an apparatus comprising logic, modules, or circuitry to perform one or more elements of the method 2200. This apparatus may be, for example, an apparatus of a base station (such as a network device 2418 that is a base station, as described herein).

Embodiments contemplated herein include an apparatus comprising: one or more processors and one or more computer-readable media comprising instructions that, when executed by the one or more processors, cause the one or more processors to perform one or more elements of the method 2200. This apparatus may be, for example, an apparatus of a base station (such as a network device 2418 that is a base station, as described herein).

Embodiments contemplated herein include a signal as described in or related to one or more elements of the method 2200.

Embodiments contemplated herein include a computer program or computer program product comprising instructions, wherein execution of the program by a processing element is to cause the processing element to carry out one or more elements of the method 2200. The processor may be a processor of a base station (such as a processor(s) 2420 of a network device 2418 that is a base station, as described herein). These instructions may be, for example, located in the processor and/or on a memory of the base station (such as a memory 2422 of a network device 2418 that is a base station, as described herein).

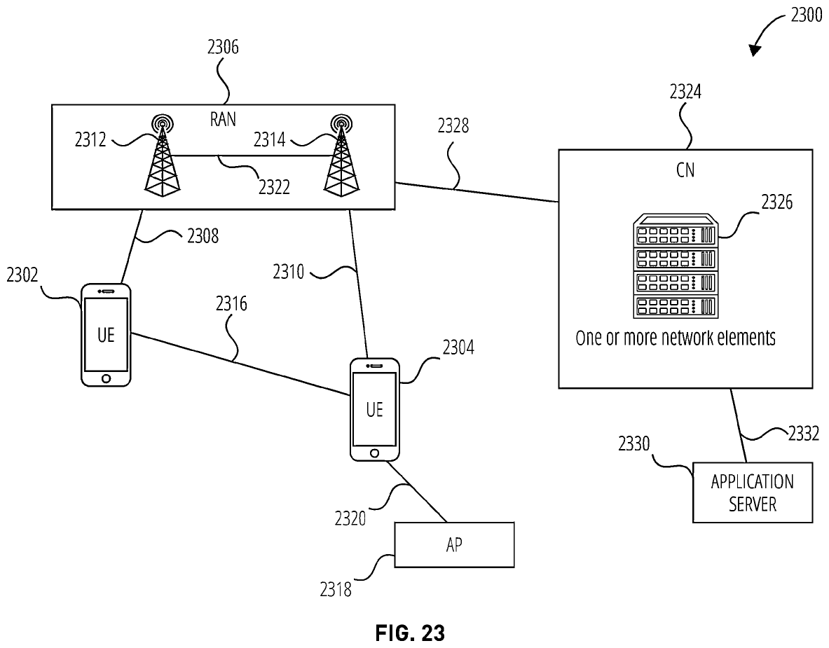

FIG. 23 illustrates an example architecture of a wireless communication system 2300, according to embodiments disclosed herein. The following description is provided for an example wireless communication system 2300 that operates in conjunction with the LTE system standards and/or 5G or NR system standards as provided by 3GPP technical specifications.

As shown by FIG. 23, the wireless communication system 2300 includes UE 2302 and UE 2304 (although any number of UEs may be used). In this example, the UE 2302 and the UE 2304 are illustrated as smartphones (e.g., handheld touchscreen mobile computing devices connectable to one or more cellular networks), but may also comprise any mobile or non-mobile computing device configured for wireless communication.

The UE 2302 and UE 2304 may be configured to communicatively couple with a RAN 2306. In embodiments, the RAN 2306 may be NG-RAN, E-UTRAN, etc. The UE 2302 and UE 2304 utilize connections (or channels) (shown as connection 2308 and connection 2310, respectively) with the RAN 2306, each of which comprises a physical communications interface. The RAN 2306 can include one or more base stations, such as base station 2312 and base station 2314, that enable the connection 2308 and connection 2310.

In this example, the connection 2308 and connection 2310 are air interfaces to enable such communicative coupling, and may be consistent with RAT(s) used by the RAN 2306, such as, for example, an LTE and/or NR.

In some embodiments, the UE 2302 and UE 2304 may also directly exchange communication data via a sidelink interface 2316. The UE 2304 is shown to be configured to access an access point (shown as AP 2318) via connection 2320. By way of example, the connection 2320 can comprise a local wireless connection, such as a connection consistent with any IEEE 802.11 protocol, wherein the AP 2318 may comprise a Wi-Fi® router. In this example, the AP 2318 may be connected to another network (for example, the Internet) without going through a CN 2324.

In embodiments, the UE 2302 and UE 2304 can be configured to communicate using orthogonal frequency division multiplexing (OFDM) communication signals with each other or with the base station 2312 and/or the base station 2314 over a multicarrier communication channel in accordance with various communication techniques, such as, but not limited to, an orthogonal frequency division multiple access (OFDMA) communication technique (e.g., for downlink communications) or a single carrier frequency division multiple access (SC-FDMA) communication technique (e.g., for uplink and ProSe or sidelink communications), although the scope of the embodiments is not limited in this respect. The OFDM signals can comprise a plurality of orthogonal subcarriers.

In some embodiments, all or parts of the base station 2312 or base station 2314 may be implemented as one or more software entities running on server computers as part of a virtual network. In addition, or in other embodiments, the base station 2312 or base station 2314 may be configured to communicate with one another via interface 2322. In embodiments where the wireless communication system 2300 is an LTE system (e.g., when the CN 2324 is an EPC), the interface 2322 may be an X2 interface. The X2 interface may be defined between two or more base stations (e.g., two or more eNBs and the like) that connect to an EPC, and/or between two eNBs connecting to the EPC. In embodiments where the wireless communication system 2300 is an NR system (e.g., when CN 2324 is a 5GC), the interface 2322 may be an Xn interface. The Xn interface is defined between two or more base stations (e.g., two or more gNBs and the like) that connect to 5GC, between a base station 2312 (e.g., a gNB) connecting to 5GC and an eNB, and/or between two eNBs connecting to 5GC (e.g., CN 2324).

The RAN 2306 is shown to be communicatively coupled to the CN 2324. The CN 2324 may comprise one or more network elements 2326, which are configured to offer various data and telecommunications services to customers/subscribers (e.g., users of UE 2302 and UE 2304) who are connected to the CN 2324 via the RAN 2306. The components of the CN 2324 may be implemented in one physical device or separate physical devices including components to read and execute instructions from a machine-readable or computer-readable medium (e.g., a non-transitory machine-readable storage medium).

In embodiments, the CN 2324 may be an EPC, and the RAN 2306 may be connected with the CN 2324 via an S1 interface 2328. In embodiments, the S1 interface 2328 may be split into two parts, an S1 user plane (S1-U) interface, which carries traffic data between the base station 2312 or base station 2314 and a serving gateway (S-GW), and the S1-MME interface, which is a signaling interface between the base station 2312 or base station 2314 and mobility management entities (MMEs).

In embodiments, the CN 2324 may be a 5GC, and the RAN 2306 may be connected with the CN 2324 via an NG interface 2328. In embodiments, the NG interface 2328 may be split into two parts, an NG user plane (NG-U) interface, which carries traffic data between the base station 2312 or base station 2314 and a user plane function (UPF), and the S1 control plane (NG-C) interface, which is a signaling interface between the base station 2312 or base station 2314 and access and mobility management functions (AMFs).

Generally, an application server 2330 may be an element offering applications that use internet protocol (IP) bearer resources with the CN 2324 (e.g., packet switched data services). The application server 2330 can also be configured to support one or more communication services (e.g., VoIP sessions, group communication sessions, etc.) for the UE 2302 and UE 2304 via the CN 2324. The application server 2330 may communicate with the CN 2324 through an IP communications interface 2332.

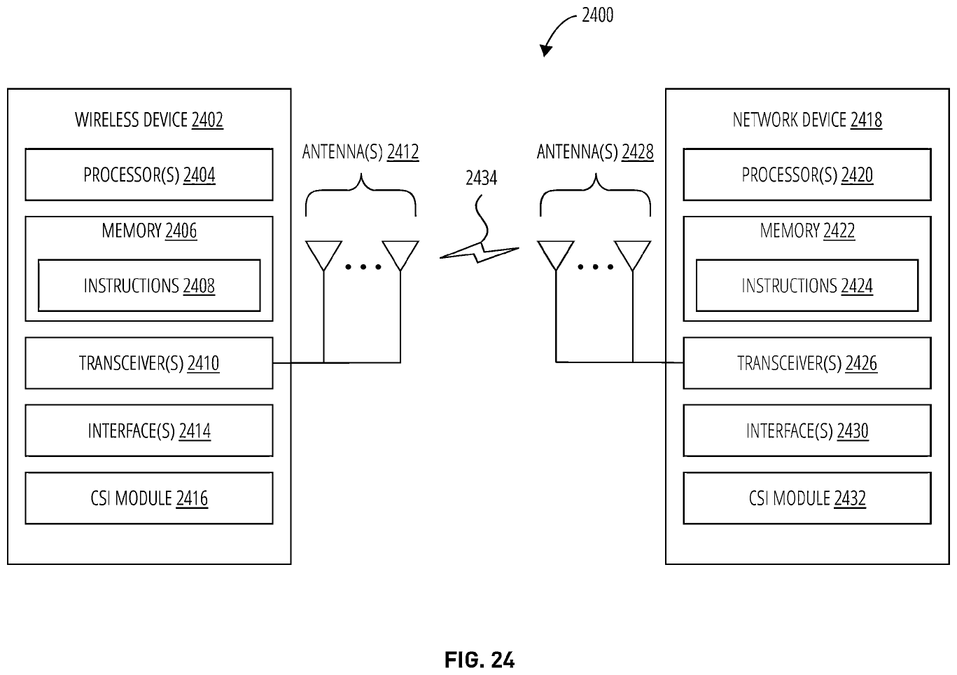

FIG. 24 illustrates a system 2400 for performing signaling 2434 between a wireless device 2402 and a network device 2418, according to embodiments disclosed herein. The system 2400 may be a portion of a wireless communications system as herein described. The wireless device 2402 may be, for example, a UE of a wireless communication system. The network device 2418 may be, for example, a base station (e.g., an eNB or a gNB) of a wireless communication system.

The wireless device 2402 may include one or more processor(s) 2404. The processor(s) 2404 may execute instructions such that various operations of the wireless device 2402 are performed, as described herein. The processor(s) 2404 may include one or more baseband processors implemented using, for example, a central processing unit (CPU), a digital signal processor (DSP), an application specific integrated circuit (ASIC), a controller, a field programmable gate array (FPGA) device, another hardware device, a firmware device, or any combination thereof configured to perform the operations described herein.

The wireless device 2402 may include a memory 2406. The memory 2406 may be a non-transitory computer-readable storage medium that stores instructions 2408 (which may include, for example, the instructions being executed by the processor(s) 2404). The instructions 2408 may also be referred to as program code or a computer program. The memory 2406 may also store data used by, and results computed by, the processor(s) 2404.

The wireless device 2402 may include one or more transceiver(s) 2410 that may include radio frequency (RF) transmitter and/or receiver circuitry that use the antenna(s) 2412 of the wireless device 2402 to facilitate signaling (e.g., the signaling 2434) to and/or from the wireless device 2402 with other devices (e.g., the network device 2418) according to corresponding RATs.

The wireless device 2402 may include one or more antenna(s) 2412 (e.g., one, two, four, or more). For embodiments with multiple antenna(s) 2412, the wireless device 2402 may leverage the spatial diversity of such multiple antenna(s) 2412 to send and/or receive multiple different data streams on the same time and frequency resources. This behavior may be referred to as, for example, multiple input multiple output (MIMO) behavior (referring to the multiple antennas used at each of a transmitting device and a receiving device that enable this aspect). MIMO transmissions by the wireless device 2402 may be accomplished according to precoding (or digital beamforming) that is applied at the wireless device 2402 that multiplexes the data streams across the antenna(s) 2412 according to known or assumed channel characteristics such that each data stream is received with an appropriate signal strength relative to other streams and at a desired location in the spatial domain (e.g., the location of a receiver associated with that data stream). Certain embodiments may use single user MIMO (SU-MIMO) methods (where the data streams are all directed to a single receiver) and/or multi user MIMO (MU-MIMO) methods (where individual data streams may be directed to individual (different) receivers in different locations in the spatial domain).

In certain embodiments having multiple antennas, the wireless device 2402 may implement analog beamforming techniques, whereby phases of the signals sent by the antenna(s) 2412 are relatively adjusted such that the (joint) transmission of the antenna(s) 2412 can be directed (this is sometimes referred to as beam steering).

The wireless device 2402 may include one or more interface(s) 2414. The interface(s) 2414 may be used to provide input to or output from the wireless device 2402. For example, a wireless device 2402 that is a UE may include interface(s) 2414 such as microphones, speakers, a touchscreen, buttons, and the like in order to allow for input and/or output to the UE by a user of the UE. Other interfaces of such a UE may be made up of transmitters, receivers, and other circuitry (e.g., other than the transceiver(s) 2410/antenna(s) 2412 already described) that allow for communication between the UE and other devices and may operate according to known protocols (e.g., Wi-Fi®, Bluetooth®, and the like).

The wireless device 2402 may include a CSI module 2416. The CSI module 2416 may be implemented via hardware, software, or combinations thereof. For example, the CSI module 2416 may be implemented as a processor, circuit, and/or instructions 2408 stored in the memory 2406 and executed by the processor(s) 2404. In some examples, the CSI module 2416 may be integrated within the processor(s) 2404 and/or the transceiver(s) 2410. For example, the CSI module 2416 may be implemented by a combination of software components (e.g., executed by a DSP or a general processor) and hardware components (e.g., logic gates and circuitry) within the processor(s) 2404 or the transceiver(s) 2410.

The CSI module 2416 may be used for various aspects of the present disclosure, for example, aspects of FIG. 1 through FIG. 22. The CSI module 2416 may be configured to, among other things, apply and use a P-CSI report configuration from a base station, apply and use a CSI report sub-configuration from a base station, and/or apply and use an M-bit CSI-RS ports bitmap from a base station, in the manner(s) described herein.

The network device 2418 may include one or more processor(s) 2420. The processor(s) 2420 may execute instructions such that various operations of the network device 2418 are performed, as described herein. The processor(s) 2420 may include one or more baseband processors implemented using, for example, a CPU, a DSP, an ASIC, a controller, an FPGA device, another hardware device, a firmware device, or any combination thereof configured to perform the operations described herein.

The network device 2418 may include a memory 2422. The memory 2422 may be a non-transitory computer-readable storage medium that stores instructions 2424 (which may include, for example, the instructions being executed by the processor(s) 2420). The instructions 2424 may also be referred to as program code or a computer program. The memory 2422 may also store data used by, and results computed by, the processor(s) 2420.

The network device 2418 may include one or more transceiver(s) 2426 that may include RF transmitter and/or receiver circuitry that use the antenna(s) 2428 of the network device 2418 to facilitate signaling (e.g., the signaling 2434) to and/or from the network device 2418 with other devices (e.g., the wireless device 2402) according to corresponding RATs.

The network device 2418 may include one or more antenna(s) 2428 (e.g., one, two, four, or more). In embodiments having multiple antenna(s) 2428, the network device 2418 may perform MIMO, digital beamforming, analog beamforming, beam steering, etc., as has been described.

The network device 2418 may include one or more interface(s) 2430. The interface(s) 2430 may be used to provide input to or output from the network device 2418. For example, a network device 2418 that is a base station may include interface(s) 2430 made up of transmitters, receivers, and other circuitry (e.g., other than the transceiver(s) 2426/antenna(s) 2428 already described) that enables the base station to communicate with other equipment in a core network, and/or that enables the base station to communicate with external networks, computers, databases, and the like for purposes of operations, administration, and maintenance of the base station or other equipment operably connected thereto.

The network device 2418 may include a CSI module 2432. The CSI module 2432 may be implemented via hardware, software, or combinations thereof. For example, the CSI module 2432 may be implemented as a processor, circuit, and/or instructions 2424 stored in the memory 2422 and executed by the processor(s) 2420. In some examples, the CSI module 2432 may be integrated within the processor(s) 2420 and/or the transceiver(s) 2426. For example, the CSI module 2432 may be implemented by a combination of software components (e.g., executed by a DSP or a general processor) and hardware components (e.g., logic gates and circuitry) within the processor(s) 2420 or the transceiver(s) 2426.

The CSI module 2432 may be used for various aspects of the present disclosure, for example, aspects of FIG. 1 through FIG. 22. The CSI module 2432 is configured to, among other things, generate a group of P-CSI report configurations and send them to a UE, indicate a particular P-CSI report configuration to a UE, generate a CSI report configuration having multiple sub-configurations and send it to a UE, indicate a particular CSI report sub-configuration to the UE, and/or generate and use an M-bit CSI-RS ports bitmap corresponding to a CSI-RS to be transmitted and indicate it to the UE, in the manner(s) described herein.

For one or more embodiments, at least one of the components set forth in one or more of the preceding figures may be configured to perform one or more operations, techniques, processes, and/or methods as set forth herein. For example, a baseband processor as described herein in connection with one or more of the preceding figures may be configured to operate in accordance with one or more of the examples set forth herein. For another example, circuitry associated with a UE, base station, network element, etc. as described above in connection with one or more of the preceding figures may be configured to operate in accordance with one or more of the examples set forth herein.

Any of the above described embodiments may be combined with any other embodiment (or combination of embodiments), unless explicitly stated otherwise. The foregoing description of one or more implementations provides illustration and description, but is not intended to be exhaustive or to limit the scope of embodiments to the precise form disclosed. Modifications and variations are possible in light of the above teachings or may be acquired from practice of various embodiments.

Embodiments and implementations of the systems and methods described herein may include various operations, which may be embodied in machine-executable instructions to be executed by a computer system. A computer system may include one or more general-purpose or special-purpose computers (or other electronic devices). The computer system may include hardware components that include specific logic for performing the operations or may include a combination of hardware, software, and/or firmware.

It should be recognized that the systems described herein include descriptions of specific embodiments. These embodiments can be combined into single systems, partially combined into other systems, split into multiple systems or divided or combined in other ways. In addition, it is contemplated that parameters, attributes, aspects, etc. of one embodiment can be used in another embodiment. The parameters, attributes, aspects, etc. are merely described in one or more embodiments for clarity, and it is recognized that the parameters, attributes, aspects, etc. can be combined with or substituted for parameters, attributes, aspects, etc. of another embodiment unless specifically disclaimed herein.

It is well understood that the use of personally identifiable information should follow privacy policies and practices that are generally recognized as meeting or exceeding industry or governmental requirements for maintaining the privacy of users. In particular, personally identifiable information data should be managed and handled so as to minimize risks of unintentional or unauthorized access or use, and the nature of authorized use should be clearly indicated to users.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive, and the description is not to be limited to the details given herein, but may be modified within the scope and equivalents of the appended claims.

1. A method of a user equipment (UE), comprising:
receiving, from a base station, a plurality of periodic channel state information (P-CSI) report configurations for providing channel state information (CSI) feedback for a component carrier (CC) used by the UE;
receiving, from the base station, an indication of a first P-CSI report configuration from the plurality of P-CSI report configurations for the CC;
generating the CSI feedback for the CC according to the first P-CSI report configuration; and
transmitting, to the base station, the CSI feedback for the CC.

2. The method of claim 1, wherein the plurality of P-CSI report configurations comprises a plurality of channel state information reference signal (CSI-RS) resource set configurations, wherein each of the plurality of CSI-RS resource set configurations is for a different number of CSI-RS ports for CSI-RS resources in a CSI-RS resource set.

3. The method of claim 1, wherein the plurality of P-CSI report configurations comprises one or more of:
a plurality of antenna panel number configurations;
a plurality of antenna array configurations;
a plurality of codebook subset restriction (CSBR) bitmap configurations;
a plurality of reporting periodicity configurations; and
a plurality of frequency granularity configurations.

4. The method of claim 1, wherein the indication of the first P-CSI report configuration is received in a medium access control control element (MAC CE) that includes a serving cell identifier (ID) information element (IE) identifying a serving cell corresponding to the CC and a P-CSI ID IE identifying the first P-CSI report configuration.

5. The method of claim 4, further comprising applying the first P-CSI report configuration after a time instance n+T_{proc}, where n is one of a first slot of a physical downlink shared channel (PDSCH) containing the MAC CE and a second slot of a physical uplink control channel (PUCCH) containing hybrid automatic repeat request acknowledgement (HARQ-ACK) signaling for the PDSCH, and where T_{proc} is a processing time for applying the first P-CSI report configuration.

6. The method of claim 1, wherein the indication of the first P-CSI report configuration is received in a scheduling downlink control information (DCI) format comprising a P-CSI ID field identifying the first P-CSI report configuration.

7. The method of claim 6, wherein the scheduling DCI format further comprises a serving cell identifier (ID) field identifying a serving cell corresponding to the CC.

8. The method of claim 1, wherein:
the indication of the first P-CSI report configuration is received in a group-common downlink control information (DCI) format with cyclic redundancy check (CRC) bits scrambled by a dedicated radio network temporary identifier (RNTI) for a plurality of UEs that includes the UE; and
a CSI block field of the UE in the group-common DCI format that corresponds to the CC identifies that the first sub-configuration is for the CC.

9. The method of claim 1, wherein the indication of the first P-CSI report configuration is received in UE-specific downlink control information (DCI) format corresponding to the UE that includes:
a serving cell identifier (ID) field identifying a serving cell corresponding to the CC;
a P-CSI field identifying the first P-CSI report configuration; and
a codebook subset restriction (CSBR) field.

10. A method of a user equipment (UE), comprising:
receiving, from a base station, a channel state information (CSI) report configuration for providing CSI feedback for a component carrier (CC) used by the UE; wherein the CSI report configuration lists a plurality of sub-configurations for generating the CSI feedback;
receiving, from the base station, an indication of a first sub-configuration of the plurality of sub-configurations;
generating the CSI feedback for the CC according to the first sub-configuration; and
transmitting, to the base station, the CSI feedback for the CC.

11. The method of claim 10, wherein the plurality of sub-configurations comprise a plurality of channel state information reference signal (CSI-RS) resource set configurations, wherein each of the plurality of CSI-RS resource set configurations is for a different number of CSI-RS ports for CSI-RS resources in a CSI-RS resource set.

12. The method of claim 10, wherein the plurality of sub-configurations comprises a plurality of codebook configurations.

13. The method of claim 12, wherein the plurality of codebook configurations comprises one or more of:
a plurality of codebook types;
a plurality of antenna panel number configurations;
a plurality of antenna array configurations; and
a plurality of codebook subset restriction (CSBR) bitmap configurations.

14. The method of claim 10, wherein the indication of the first sub-configuration is received in a medium access control control element (MAC CE) that includes a serving cell identifier (ID) information element (IE) identifying a serving cell corresponding to the CC and a sub-configuration indicator IE identifying the first sub-configuration.

15. The method of claim 14, further comprising applying the first sub-configuration after a time n+T_{proc}, where n is one of a first slot of a physical downlink shared channel (PDSCH) containing the MAC CE and a second slot of a physical uplink control channel (PUCCH) containing hybrid automatic repeat request acknowledgement (HARQ-ACK) signaling for the PDSCH, and where T_{proc} is a processing time for applying the first sub-configuration.

16. The method of claim 10, wherein the indication of the first sub-configuration is received in a scheduling downlink control information (DCI) format that includes a sub-configuration indicator field identifying the first sub-configuration.

17. The method of claim 16, wherein the scheduling DCI format further comprises a serving cell identifier (ID) field identifying a serving cell corresponding to the CC.

18. The method of claim 10, wherein:
the indication of the first sub-configuration is received in a group-common downlink control information (DCI) format with cyclic redundancy check (CRC) bits scrambled by a dedicated radio network temporary identifier (RNTI) for a plurality of UEs that includes the UE; and
a CSI block field of the UE in the group-common DCI format that corresponds to the CC identifies that the first sub-configuration is for the CC.

19. The method of claim 10, wherein the indication of the first sub-configuration is received in UE-specific downlink control information (DCI) format corresponding to the UE that includes:
a serving cell identifier (ID) field identifying a serving cell corresponding to the CC;
a sub-configuration indicator field identifying the first sub-configuration; and
a codebook subset restriction (CSBR) field.

20. The method of claim 10, wherein the indication of the first sub-configuration is received in an aperiodic CSI (A-CSI) report triggering downlink control information (DCI) for the CSI feedback that includes a sub-configuration indicator field identifying the first sub-configuration.

21-30. (canceled)
